(19) United States
(12) Patent Application Publication
(10) Pub. No.: US 2022/0338929 A1
(43) Pub. Date: Oct. 27, 2022

(54) Decision Support Tool for Endovascular Procedures

(71) Applicant: Mentice AB, Gothenburg (SE)

(72) Inventors: Mayank Goyal, Calgary (CA); Johanna Ospel, Lorrach (DE)

(21) Appl. No.: 17/660,339

(22) Filed: Apr. 22, 2022

Related U.S. Application Data
(60) Provisional application No. 63/179,924, filed on Apr. 26, 2021.

(51) Int. Cl. A61B 34/10; G16H 20/40

(52) U.S. Cl. CPC A61B 34/10; G16H 20/40; A61B 2034/105; A61B 2034/107; A61M 2025/0166

(57) ABSTRACT

The invention relates to systems and methods for assisting a physician in making treatment decisions and to assist in the planning of endovascular surgical procedures. In particular, the invention relates to systems and methods for helping a physician decide if access to the cervical and cerebral arteries is best achieved via a radial artery or femoral artery access route (or other) based on objective assessment of the likelihood of success by a specific access point and having consideration to the available endovascular equipment and a particular patient's anatomy.

### FIELD OF THE INVENTION

The invention relates to systems and methods for assisting a physician in making treatment decisions and to assist in the planning of endovascular surgical procedures. In particular, the invention relates to systems and methods for helping a physician decide if access to the cervical and cerebral arteries is best achieved via a radial artery or femoral artery access route (or other) based on objective assessment of the likelihood of success by a specific access point and having consideration to the available endovascular equipment and a particular patient's anatomy.

### BACKGROUND OF THE INVENTION

The human body has an extensive network of blood vessels including both the venous and arterial systems for circulating blood throughout the body as a whole as well as the organs of the body.

In recent decades, various surgical procedures involving traumatic and highly invasive access to the body have been replaced with procedures that involve the use of one or more catheters being introduced in a minimally invasive fashion through a small skin nick into and advanced through the vascular system of the body. These endovascular procedures are highly effective to diagnose and/or to treat diseases involving the vasculature of a particular organ without requiring large incisions and/or deep surgeries through other tissues to complete the procedure.

For example, strokes (e.g. ischemic strokes caused by blood clot blockages of brain vessels), coronary artery blockages within the heart and various heart defects may be treated by advancing catheters through the vasculature to the affected site where various diagnostic and treatment procedures can be initiated to identify and treat the problem

Well known procedures include the deployment of stents via a catheter into an occluded vessel (both coronary and cerebral). Catheter procedures are also done in other parts of the body including leg vessels, renal arteries and other complex vascular percutaneous vascular procedures including treatment of valvular heart disease, aortic dissections, dysrhythmias and management of shunts for dialysis patients. Similarly, complex aneurysms in the brain and other locations are increasingly treated via a percutaneous endovascular route.

In order to effectively use catheters within the body to complete a medical procedure, generally the catheters must be flexible enough to follow through the tortuous curves of the body's vascular system whilst being stiff enough to gain and hold position during the procedure. This can include the steps of advancing one or more catheters to the desired site as well as the steps of completing a procedure including passing tools through a catheter to the desired site once access to the site has been gained.

As is known, the parameters and variables of the human vasculature including the diameter of vessels, the curves of the vessels and, the angles of vessels relative to one another, catheters and associated equipment are provided with a wide range of properties to enable procedures to be completed.

For example, if the catheter is too flexible, the catheter may fall back into other vessels within the vascular system. If the catheter is too stiff, it may cause damage to the surrounding tissue as it navigates through curves of the vessels, if it is able to be moved at all. In both instances, the use of an ineffective catheter may cause significant time delays in completing the procedure.

Importantly, in certain endovascular interventions and specifically procedures to remove a blood clot from the brain of a patient who has had an ischemic stroke, “time is brain”, meaning that delays in completing a procedure can significantly affect the outcome for the patient.

Importantly, the degree of tortuosity within blood vessels as well as the stiffness of vessels increases with age due to multiple factors including atherosclerotic disease, loss of height of the spine, etc. can affect the ease of completing a procedure. With improving technologies, more and more of these procedures are being done in an older population despite the increased complexity of conducting procedures through tortuous and/or stiffer vessels.

That is, it is well known that there are significant variations in the vascular anatomy of patients with the result being that in some patients, navigation of endovascular equipment from one point to another is straightforward, whereas in others may be impossible with available equipment.

To take a typical catheter procedure as an example and as described in more detail below, to access the blood vessels in the head, the interventionist typically navigates a catheter system up the descending aorta from the femoral artery into the aortic arch and into the left common carotid artery. For the purposes of the description herein a “catheter system” implies various combinations of inner catheters (e.g. diagnostic catheters, guide wires, microcatheters) and outer guide catheters (e.g. distal access catheters and balloon guide catheters) where the inner and outer components are substantially coaxial and can slide over or within the other. This can include coaxial, triaxial and quadra-axial procedures. In most circumstances, the components will move together with a guide wire usually extending beyond the outer guide catheter and inner components such as a diagnostic catheter or microcatheter. Hence, the catheter system may be both a combination of a wire within the catheter during antegrade movement of the catheter system but may also mean just the catheter without the wire. Antegrade movement is generally conducted by a combination of advancing the guide wire followed by advancing the catheter over the wire, all of which may involve twisting or turning the catheter and wire in order to turn the distal end of the guide wire and catheter into the appropriate vessel. After reaching the aortic arch, for example, the catheter system is navigated up the left common carotid artery and into the left internal carotid artery. Depending on the underlying condition and the procedure being conducted, at this stage the interventionist may utilize a variety of different catheters (including microcatheters and microwires) and techniques to gain access to intracranial vessels and ultimately to the site where the procedure is to be conducted.

A stiff catheter system can straighten out tortuous vessels (which may or may not be advantageous) and/or damage the vessel as it is being navigated through a tight curve. However, if the catheter is very flexible, it may not be able to maintain its position within the vessel and, for example, fall back into the aortic arch after it has been successfully guided into the left internal carotid artery (especially as further catheters and tools are being advanced through the catheter to enter the brain vessels and/or as the guide wire is withdrawn). In some cases, additional catheters face friction as they are being advanced and this creates a backward force on the guiding catheter hence preventing the interventionist from completing a procedure and/or wasting time in removing a catheter and selecting and navigating a different catheter into position.

### Femoral Artery Vs Radial Artery Access

Femoral artery access has been the preferred access point for cerebral artery procedures since the initial development of endovascular procedures, because of the relatively large size of the artery and hence the ability to introduce catheters of a larger diameter. In addition, the artery is quite superficial and easy to compress against the head of femur after the procedure, to stop the bleeding at the end of the procedure. In addition, access from the femoral artery over the aortic arch into the cervical vessels often has favorable, non-acute angles that allow quicker access into the cervical vessels without catheters getting kinked.

The downside of the femoral artery as an access point is that A) it can be relatively deep in obese patients and B) if the site of puncture is slightly high, bleeding from the artery can go into the retroperitoneum and is difficult to stop with compression. Hence, the incision to gain access to the femoral artery is deeper as compared to the radial artery, carries a larger bleeding risk and requires longer post-operative care (with that patient being confined to their bed for several hours) to monitor the incision prior to patient discharge from the care facility and thus typically incurs higher costs.

In comparison, the radial access point provides various advantages including limited need for post-operative monitoring of the incision and thus more rapid discharge of the patient from the care facility. However, while favorable not all procedures can be completed via a radial access.

Access from the radial artery provides two main challenges, namely the small size of the radial artery which limits the size of catheters that can be introduced and different, and usually more acute access angles at or near the aortic arch from this direction.

### Catheter Design and Performance

As noted above, two classes of catheters used in cerebral procedures are diagnostic and guide catheters. Diagnostic catheters are generally those used to gain access to an area of interest whereas guiding catheters are used to support and guide additional equipment including diagnostic catheters, guidewires balloons, other catheters etc. as may be required for a particular surgical technique.

Typical diagnostic catheters will range from 4F to 6F (French) and have lengths of 65-125 cm. They may have braided wall structures and they will generally have a soft tip with a range of shapes formed into the tip.

Guide catheters are generally larger (e.g. 6-8F) and are 80-100 cm in length. They generally have reinforced construction with a significantly stiffer shaft to provide back-up support and stability for the advancement of any additional equipment as mentioned above.

From an anatomical perspective, catheters generally pass through different zones of the vasculature, namely the abdominal and thoracic vasculature between the femoral artery and aortic arch (approximately 50-75 cm), the cervical vasculature (approximately 15-20 cm) and the cephalic/cerebral vasculature (approximately 10-15 cm), with vessel diameters constantly changing and being smallest in the cerebral vasculature.

Various properties and geometries may also be engineered into both diagnostic and guide catheters including:
a) Trackability—the ability of the catheter to slide over a guide wire particularly through tortuous (tightly curved) vessels.
b) Pushability—the ability to advance the tip or head of the catheter based on the input from the operator from the hub (i.e. from outside the body).
c) Torquability—the ability to steer the tip of the catheter based on twisting at the hub by the operator.
d) Tip or head shape—the shape of the tip or head of the catheter will assist the operator in navigating the distal tip of the catheter through particular anatomical features. For example, a catheter may have a straight, simple curve, complex curve, reverse curve or double curve shapes inter alia. Such shapes may be categorized as simple or complex.

In particular, diagnostic catheters are provided with a wide range of tips having the above shapes to allow the surgeon to choose an optimal tip shape that is best for an individual patient's anatomy when conducting a procedure.

### Catheter Construction

Each catheter may be constructed from a plurality of materials, having various structures and/or layers within the catheter wall structure to give the catheter particular properties or functional characteristics. These may include:
a) Surface Coatings—Surface coatings desirably reduce thrombogenicity, may have low friction coefficients and/or anti-microbial characteristics.
b) Reinforcement-Internal wire braiding is used to impart torque control/stiffness characteristics to the catheter.
c) Polymer Layers—Different polymers may be used to give different structural characteristics to the body of the catheter. For example,
   i. Polyurethanes can be soft and pliable and hence follow guide wires more effectively. However, they have a higher coefficient of friction.
   ii. Nylon may be used for stiffness and is able to tolerate higher flow rates of fluids through them.

The choice of a particular catheter or system of catheters may be determined by the skill and experience of a particular surgeon.

Typical properties of different catheters are summarized in Table 1.

TABLE 1
Summary of Catheter Properties

| | | | | Typical Tip |
|---|---|---|---|---|
| Catheter | Body Properties | Diameter | Typical Length | Features |
| Guide | Usually quite | 6-8F | Extracorporeal + | May have a |
| Catheter | stiff | | Groin to Carotid | balloon at |
| | Atraumatic | | 80-100 cm | its tip |
| | tip | | | |
| | Supports and | | | |
| | guides other | | | |
| | catheters | | | |
| | Double | | | |
| | lumen if | | | |
| | Balloon | | | |
| | Guide | | | |
| | Catheter | | | |
| | (BGC) | | | |
| Diagnostic | Variable Tip | 4-6F | Extracorporeal + | Soft Tip |
| Catheter | Stiffness | | Groin to Carotid | Multiple |
| | Variable Tip | | 100-125 cm | Shapes |
| | Shapes | | | |
| | Torquable | | | |
| Micro- | Soft Tip | 1-5-2.5F | Goes through the | Rounded |
| catheter | Pushable | | guide catheter | convex end |
| | Trackable | | Travel to | Soft Tip |
| | | | intracranial | |
| | | | vessels (over a | |
| | | | microwire) and | |
| | | | beyond blood | |
| | | | clots or into | |
| | | | aneurysms (sac- | |
| | | | like bulges of the | |
| | | | vessel wall). | |
| | | | 150 cm | |
| Guide | Pushable | 1F | Travels inside of a | Pre-formed |
| Wire | Torquable | | diagnostic | shaped |
| | | | catheter or guide | |
| | | | catheter (serves | |
| | | | as a guiding | |
| | | | structure for these | |
| | | | catheters and is | |
| | | | used to advance | |
| | | | them to the | |
| | | | cervical carotid | |
| | | | artery) | |
| | | | 150-300 cm | |
| Aspiration | Multizone | 4-7F (diameter | Travel inside the | Rounded |
| Catheter | (may be up | may be more | guide catheter. | convex end |
| | to 12-15 | proximally to | Usually over a | Soft Tip |
| | zones) | allow for better | microcatheter | Challenging |
| | Increasing | suction. | Extracorporeal + | design to |
| | level of | | Groin to Occlusion | prevent |
| | softness | | 105-125 cm | collapse of |
| | distally to | | | the lumen |
| | allow the | | | during |
| | catheter to | | | passing |
| | negotiate | | | through |
| | significant | | | significant |
| | tortuousity | | | curvature |
| | and remain | | | and while |
| | atraumatic | | | applying |
| | Distal | | | suction |
| | transition | | | |
| | zones may | | | |
| | extend for | | | |
| | 30-40 cm) | | | |
| | Enables two- | | | |
| | way Fluid | | | |
| | Flow | | | |
| | Pushable | | | |
| Stent | Integrated | Very small in its | Extracorporeal + | |
| | Clot Retrieval | collapsed state | Groin to Occlusion | |
| | System | (travels through | 180 cm | |
| | Pushable | microcatheter). | | |
| | | In expanded | Travels through | |
| | | state: 3-6 mm | microcatheter | |
| Microwire | Pushable | 180-200 cm | Extracorporeal to | Round soft |
| | Torquable | Travels through | intracranially | tip |
| | 10-16/1000 | microcatheter | (beyond the clot) | |
| | of an inch | | | |

### SUMMARY OF THE INVENTION

In accordance with the invention, systems for enabling analysis of a patient's vasculature and providing output to physicians in conducting endovascular procedures are described.

In a first aspect, a system for analyzing tortuosity of a patient vasculature to provide input to a physician preparing for an endovascular surgical procedure is described, the system comprising: a database having a plurality of past patient image records (PPIR) wherein each PPIR includes a visual representation of a patient's vasculature, the database enabling access and assessment by one or more experts and wherein each PPIR can be updated to include one or more success scores where a success score is a rating of difficulty for completing an endovascular procedure (EP) access from an access point to a target vessel.

In various embodiments, the system includes a current patient input system for uploading a current patient image record (CPIR); a comparison system for comparing the CPIR to the plurality of PPIRs to determine a closest match between one or more PPIRs and the CPIR; and a success score display system for displaying one or more success scores from the closest match.

In another embodiment, the database includes separate success scores from two or more access points.

In various embodiments:
the two or more access points include a radial artery access point and a femoral artery access point.
the target vessels include any one of or a combination of right vertebral artery (RVA), right common carotid artery (RCCA), brachiocephalic trunk (BCT), left common carotid artery (LCCA), and left vertebral artery (LVA).

In another embodiment, the system further includes:
a vessel measurement system, the vessel measurement system enabling measurement of vessel branch point angles from different access points.
a vessel tortuosity measurement system (VTMS), the VTMS enabling measurement of zones of interest in a PPIR and CPIR and calculation of one or more tortuosity measures in a zone of interest.

In one embodiment, the VTMS enables identification of one or more of vessel apex, vessel inflection point, vessel segment length, branch point angle, vessel support zone and vessel unsupported zone in 3D space.

In various embodiments, the VTMS enables identification of one or more of vessel looping, vessel kinking, vessel coiling, vessel corkscrew in 3D space and/or tortuosity measure includes sum of angles (SOAM), tortuosity index (TI), and curvature metric (CM).

In another embodiment, the success score further includes a risky manoeuvre (RM) output score and where the RM output score includes any one of or a combination of a measure of risk of local injury to a blood vessel and risk of dislodging a plaque or thrombus.

In one embodiment, the success score further includes a time factor score representing a time to complete an EP from any one of or a combination of a radial artery or femoral artery access point to a target vessel.

In one embodiment, the system further includes a vasculature modelling system (VMS) enabling modelling of the vasculature in 3D space where the VMS utilizes PPIRs and/or CPIRs and where the VMIS determines 3D surface coordinates for vessel walls, vessel centerlines, branch point angles and vessel apexes.

In one embodiment, the vasculature modelling system calculates tortuosity parameters for a modelled vasculature from the 3D surface coordinates for vessel walls, vessel centerlines, branch point angles and vessel apexes.

In another embodiment, the system includes an endovascular equipment (EE) database and where each PPIR can be updated to include recommended EE to complete an EP from one or more access points to one or more target vessels.

In another embodiment, the system includes a recommended EE display system for displaying an output of one or more pieces of EE recommended to conduct a procedure.

In another embodiment, the EE database enables a user to filter for available EE at a treatment facility and update success scores based on available EE.

In another embodiment, the system further comprises a hooked catheter reform module (HCRM) where the HCRM calculates vessel volumes within defined vessel segments and determines, based on physical size parameters of a hooked catheter, if the hooked catheter can be reformed in one or more vessel segments.

In another embodiment, the EE database includes modelled parameters of EE and the system further comprises an EE advancement module (EEAM) enabling simulation of EE advancement within a modelled vasculature wherein modelled EE is progressively advanced within a modelled vasculature and the EE advancement module tests progressive movement of modelled EE within the modelled vasculature to determine if the modelled EE can be advanced based on the modelled parameters.

In another embodiment, the EEAM includes an output module to display the feasibility of advancing specific EE within a vasculature.

In another embodiment, the EEAM output module displays color coded zones within a modelled vasculature and where a displayed color represents relative feasibility of advancing EE through a zone of the modelled vasculature.

In another embodiment, the EE database includes EE physical dimension and performance parameters for different EE, the EE selected from any one of a combination of guide wires, diagnostic catheters, guide catheters and stents.

In another embodiment, performance parameters include any one of or a combination of stiffness and torqueability.

In another embodiment, the EE database includes physical dimension and performance parameters for one or more combinations of guide wires and diagnostic catheters.

In another embodiment, the EE database includes physical dimension and performance parameters for one or more combinations of guide wires, diagnostic catheters and guide catheters.

In another embodiment, the EEAM evaluates the feasibility of advancing a guide catheter over a combined guide wire and diagnostic catheter based on a combined stiffness of each of the guide wire, diagnostic catheter and guide catheter.

In another embodiment, the past patient database includes a questionnaire module enabling experts reviewing PPIRs to assign success scores to a past patient image record.

In another embodiment, the system enables a training physician to access the PPIRs to review the success scores and EE used in past EPs.

In another embodiment, each PPIR is assembled into a PPIR 3D model and the system further includes a PPIR parameter measurement module for determining any one of or a combination of branch points, apex points, branch point distances and apex point distances.

In another embodiment, each CPIR is assembled into a CPIR 3D model and the system further includes a CPIR parameter measurement module for determining any one of or a combination of branch points, apex points, branch point distances and apex point distances.

In another embodiment, the system includes a comparison module where any one of or a combination of the branch points, apex points, branch point distances and apex point distances from the PPIR 3D models and a CPIR 3D model are compared in 3D space to identify one or more PPIR 3D models most closely matching the CPIR 3D model.

In another aspect, the invention provides a system for analyzing a patient vasculature to assign a success score for completing an endovascular procedure (EP) from an access point to a target vessel, the system including: a database having a plurality of past patient image records (PPIR) wherein each PPIR includes a visual representation of a patient's vasculature and success scores assigned to each PPIR; a PPIR analysis module for calculating a success score for a current patient image record (CPIR) wherein the PPIR analysis module calculates branch point angles and vessel tortuosity between the access point and the target vessel of CPIR and compares the branch point angles and vessel tortuosity of the CPIR to branch point angles and vessel tortuosity of PPIRs to obtain a best fit to the current patient and assign a success score based on the best fit.

### DESCRIPTION OF THE DRAWINGS

The invention is described with reference to the drawings in which:

FIG. 1A is a schematic diagram of a typical aortic arch anatomy shown branch angles from a radial artery access route.

FIG. 1B is a schematic diagram of a typical aortic arch anatomy shown branch angles from a femoral artery access route.

FIG. 1C is a schematic diagram of a tortuous aortic arch anatomy shown branch angles from a radial artery access route.

FIG. 1D is a schematic diagram of a tortuous aortic arch anatomy shown branch angles from a femoral artery access route.

FIG. 2A is a schematic diagram illustrating how Tortuosity Index is measured.

FIG. 2B is a schematic diagram illustrating how Sum of Angles is measured.

FIG. 2C is a schematic diagram illustrating how Curvature Metric is measured.

FIG. 2D (A) and (B) are representative 3D models of a patient's aorta illustrating marking/calculation of representative fixed anatomical points (FAPs) (A) and volume segments (B) within vessels.

FIGS. 3A and 3B are schematic diagrams showing differences in branch angles from radial artery access vs. femoral artery access in one patient anatomy.

FIGS. 4A and 4B are schematic diagrams showing differences between radial artery access vs. femoral artery access in one patient anatomy having a tortuous section.

FIGS. 5A-5D illustrate the process of straightening and un-straightening a typical hooked diagnostic catheter.

FIGS. 5E and 5F illustrate typical procedural steps for hooking a target vessel from a femoral artery and radial artery access.

FIGS. 5G(A-D), 5H(A-D), 51(A-E) and 5J(A-F) are sketches of an aortic arch that each illustrate how advancing individual EE can cause EE dislocation from a target vessel at different steps of a procedure.

FIG. 6 is a flowchart illustrating a process by which general success scores for a current patient may be determined using expert review of past patient images.

FIG. 6A is a 3D model of a tortuous aortic arch anatomy.

FIG. 6B is a flowchart illustrating a process by which a tortuosity score and recommended EE output for a current patient may be determined using expert review of past patient images.

FIG. 6C is an overview illustrating how a model of a patient's anatomy may be marked and measured to determine branch angles.

FIG. 6D (A-C) is a schematic representation of the relationships between the anatomies of different patients that can be utilized as a basis for comparison for obtaining prediction outcomes.

FIGS. 6E and 6F are representative outputs illustrating relative degrees of difficulty of navigating EE through two different vessel anatomies including RA and FA access routes in accordance with various embodiments of the invention.

FIG. 6G is a 3D model of a patient's anatomy illustrating difficult RA access.

FIG. 7 is a flowchart illustrating a process by which a tortuosity score and recommended EE output for a current patient may be determined using expert review of past patient images and access to a EE database.

FIG. 7A is a flowchart illustrating a process by which a likelihood of success score, zones of interest, tortuosity score and recommended EE output for a current patient may be determined using automated review of past patient images and EE database information.

FIG. 8 is a flowchart illustrating a process by which output scores are obtained from patient data, target vessel and EE inputs.

FIG. 9 is representative overview showing structural equation modelling (SEM) inputs and outputs for one embodiment of the invention.

FIG. 10 is a flowchart illustrating a process for introducing feedback information back into the models to enable model improvement.

### DETAILED DESCRIPTION OF THE INVENTION

### Terms

For the purposes of describing the invention, the following definitions and terms apply to the description:
Endovascular Equipment (EE) generally refers to all guide wire (GW), diagnostic catheter (DC), guide catheter (GC), balloon guide catheter (BGC), microcatheter (MC), microwire (MW) equipment, stent-retrievers and other related equipment used to conduct endovascular procedures. EE may include peripheral equipment including pumps and sheath equipment.
Endovascular Procedures (EPs) are minimally invasive procedures conducted using endovascular equipment through vessels of the body. Access to the blood vessel is established via a puncture through the skin.
Catheters are pieces of endovascular equipment having a structure that includes a hollow lumen. Other EE can be advanced through this lumen.
Wires are endovascular equipment without a hollow lumen that are generally used to navigate, advance and support catheters.
Catheter System(s) is/are combinations of endovascular equipment that are operatively combined with one another and utilized in combination with other equipment in a generally coaxial manner. For example, a catheter system may be a combination of a guide wire that is inserted into a diagnostic catheter (a bi-axial system) or a combination of a guide wire that is inside a diagnostic catheter, which are then inserted in a guide catheter (a tri-axial system).
Vessel branch point is a point of branching of two arterial vessels where one vessel gives rise to two or more vessels (the tip of the catheter and/or wire can go into any of the branches).
An Apex point is a point on a curve mid-way between two boundary or end points.
An Inflection point is a point within a vessel where the curvature of the vessel changes from concave/convex in one direction to concave/convex in the opposite direction.
Vessel curvature is the curvature of a vessel based on an average radius of a curve through the centreline of the vessel.
A C-shaped curve is a curve that is curved in one direction generally having one common radius, a curve apex and two boundary or end points.
An S-shaped curve is a curve that is curved in two directions having more than 1 curve apex. S-shaped curves may be characterized according to their amplitude and frequency. For example, s-shaped curves may be defined as high amplitude, low frequency (Type A), medium amplitude-medium frequency (Type B) or low amplitude, high frequency (Type C) (see 10.1161/HypertensionAHA.118.11647).
Kinking is defined as acute vessel angulation ranging in severity from mild (angle greater than 60 degrees) to moderate (30-60 degrees) to severe (less than 30 degrees).
Looping is defined as an exaggerated s-shaped curve.
Coiling is defined as a circular course where a vessel has a 360 degree turn.
Corkscrew is defined as a helical course along the length of the vessel.
Tortuosity Index (TI) is the ratio between the length along the centerline of a vessel and the linear distance between two end points.
Sum of Angles (SOA) is the ratio between the sum of all angles at the inflection points along the centerline of a vessel and the length of the centerline.
Curvature metric (CM) is the number of curves along a vessel multiplied by the length of the centerline and divided by the distance between end points.
Vessel take-offs/vessel branch points (synonymous) are points within a vessel where a parent vessel branches into two daughter vessels. In this application, “vessel” exclusively refers to arterial vessels.
A Supported Vessel Zone is a vessel zone that has continuous vessel walls along a particular path (e.g. through a curvature or inflection point) and a circular, similar cross section (i.e. no major change in diameter) along the path wherein forward pressure on the catheter and/or wire is likely to result in forward movement of the catheter/wire tip.
An Unsupported Vessel Zone is a vessel zone that does not have continuous vessel walls along a particular path and has a non-circular cross section, possibly with changes in cross section along a path. For example, for a vessel curvature that includes a vessel branch point, the region of the vessel curvature that passes through a vessel branch will have a zone that is unsupported and that has a non-circular cross-section, with its diameter often decreasing immediately at the vessel branch point. In this case, forward pressure on the catheter and/or wire may not result in corresponding forward movement of the catheter/wire tip with build-up of catheter/wire in the unsupported zone.
Take-off-angle is the angle between the intersection of the central axis of the parent vessel and the central axis of one of the daughter vessels at a vessel branch point. As the central axis of a parent vessel and a daughter vessel may be curves, the take-off angle may be determined from a point on the central access of the parent vessel or daughter vessel, a short distance (e.g. 1-2 cm) from a vessel branch point.
Tortuosity is an umbrella term describing vessel morphology including vessel curvatures, including branch-points, c- and s-shaped curves, inflection points and kinked or coiled vessels, and may be a measure of resistance to forward movement of EE through supported and unsupported vessels including movement through branch-points, c- and s-shaped curves, inflection points and kinked or coiled vessels.
Cervical (access) vessels are the great vessels branching off the aortic arch (RSA, BCT, LCCA, LSA) enabling access to the vessels supplying the brain.
Procedure time-sensitivity is a measure of how fast a neuroangiographic procedure needs to be completed. A time-sensitive procedure is thrombectomy for acute ischemic stroke since irreversible brain parenchymal damage in acute ischemic stroke is progressing quickly. Non-time-sensitive procedures are typically diagnostic angiographies or elective coiling of unruptured aneurysms.
Risky maneuver (RM) is a maneuver where there is a higher risk of complication broadly divided into two categories:
   a) risk of local injury to the blood vessel, e.g. produce a dissection; or
   b) risk of dislodging plaque and/or thrombus, resulting in a stroke.
As discussed in more detail below, it is understood that there is an overall risk to any procedure based on patient-specific factors such as age and atherosclerotic burden and based on operator factors such as operator skillset and experience. These patient- and operator-specific risk factors are not taken into account when using the term RM. As such, RM refers to risks that can be attributed to how certain procedures and procedure steps are performed. If a certain way of performing a specific procedure step (e.g. obtaining radial access) has a higher risk of causing complications (of either category a) or b)) compared to an alternative way of performing this procedure step (e.g. obtaining femoral access), it is considered a RM. While patient and operator-specific factors are not included in the definition of RM, it is expected that these factors in addition to other factors, such as time sensitivity of the procedure, will influence whether a procedure/step is considered an RM. That is, under certain circumstances and given certain patient characteristics, a certain procedure step might be an RM while under other circumstances, it might be safe to do and thus not be a RM. In the following, RMs are graded into low/moderate/high RM, depending on the likelihood they cause a complication. Initially, and as explained below, RMs will be classified as low/moderate/high based on expert opinion. As more data has been collected and the data within databases becomes sufficiently large, RM ranking will be assigned based on predicted complication risks that are derived from the past patient database.

### Aortic Arch Anatomy and Variables

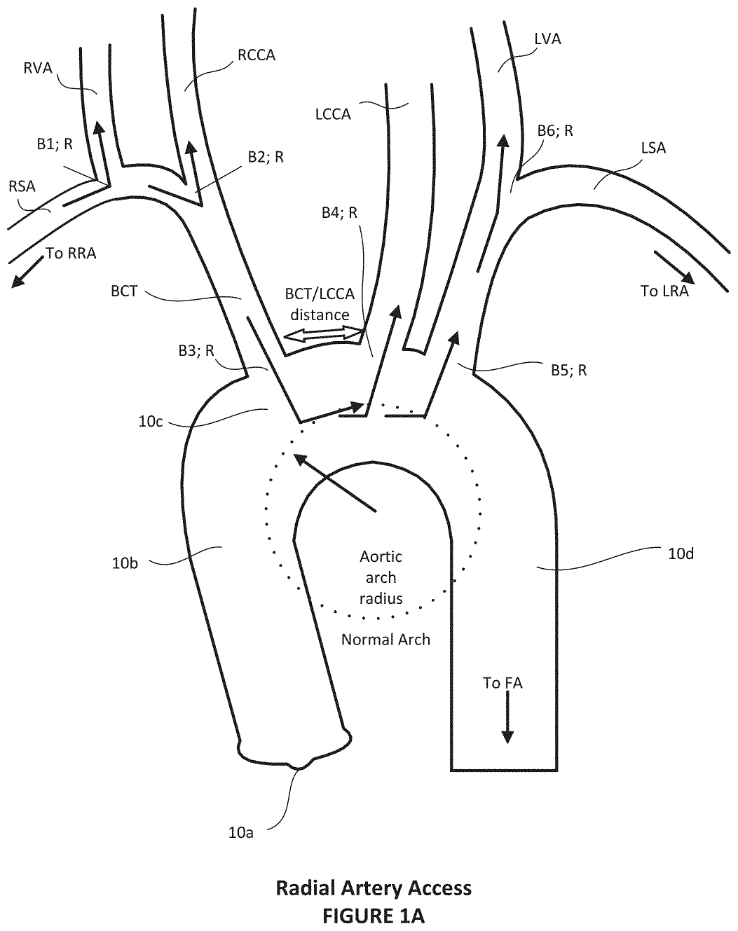

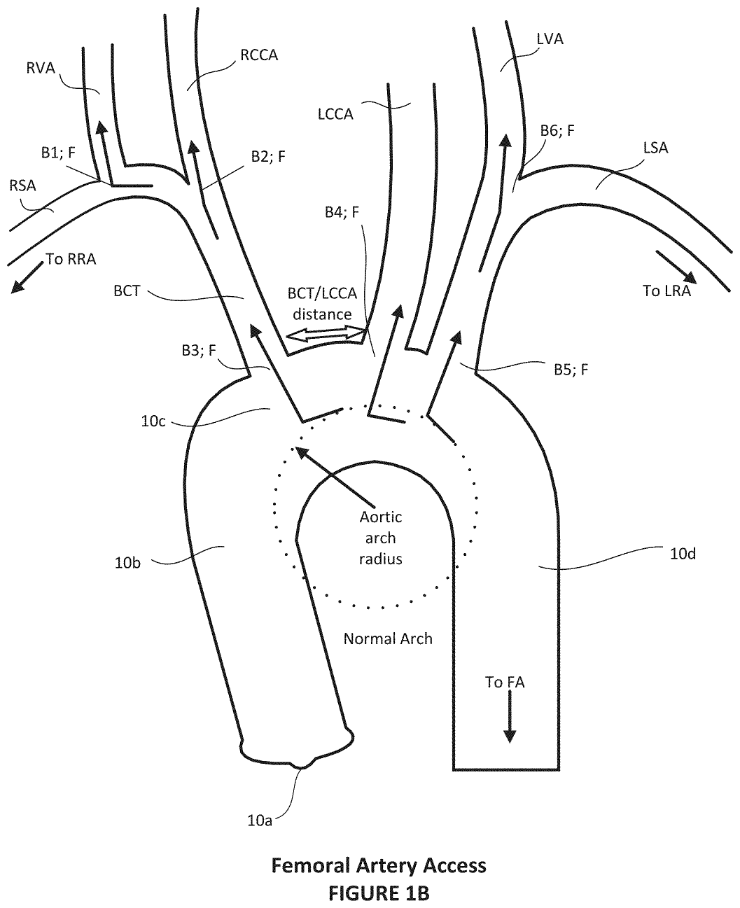

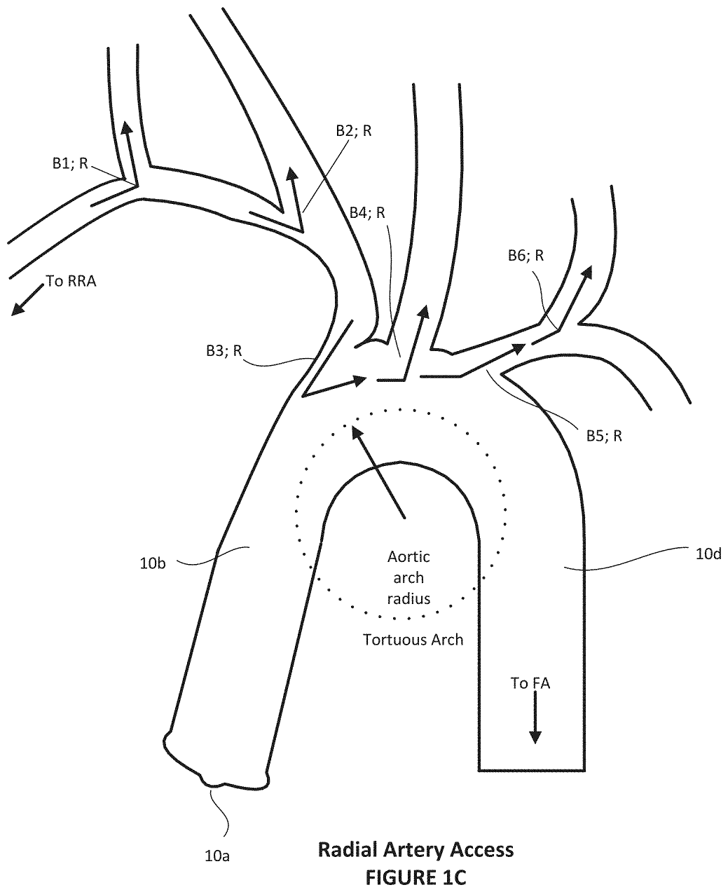

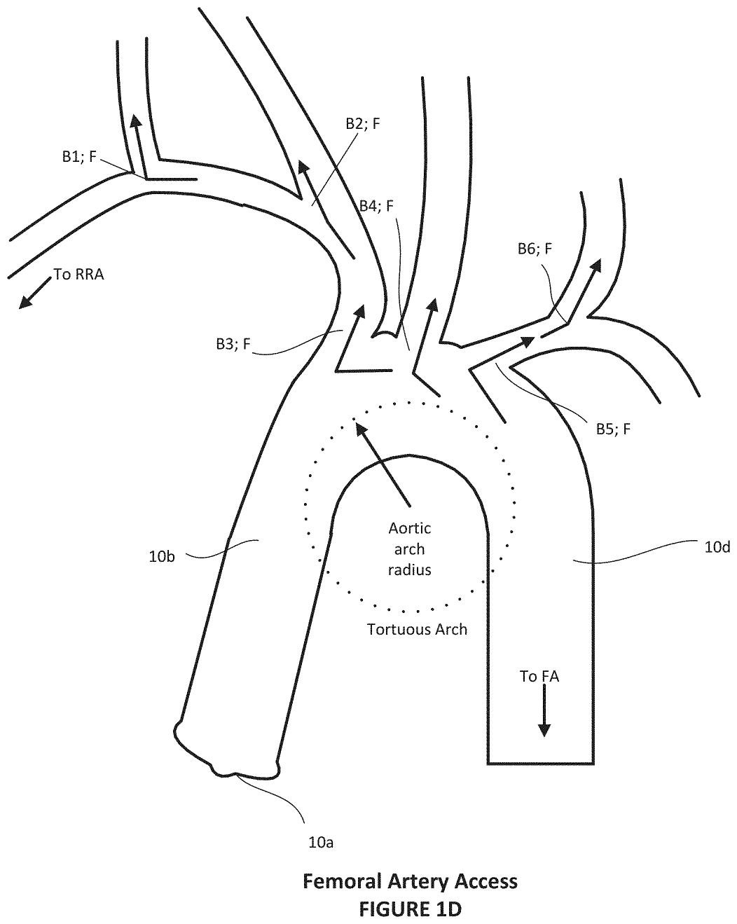

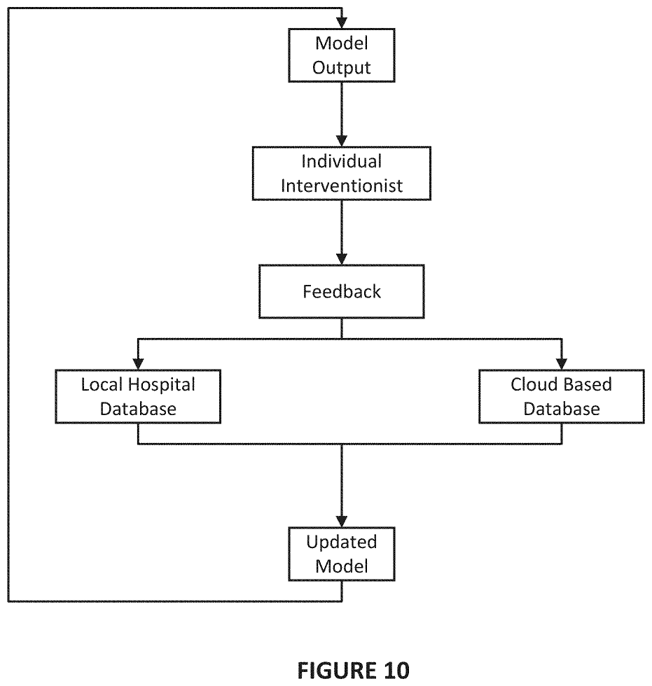

FIGS. 1A and 1B show a schematic approximate frontal (so-called “coronal”) plane cross-section of a typical normal, non-tortuous aortic arch as it may be encountered in a young patient without degenerative vessel changes. FIGS. 10 and 1D show a similar frontal plane cross section of a tortuous aortic arch, as it is often encountered in older patients with diseased vessels. As shown in each figure, the aortic arch generally extends from the aortic valve 10a and includes the ascending aorta 10b, aortic arch 10c and descending aorta 10d. For the purposes of general description and describing various vessel branch points, major vessels branching off the aortic arch and referenced herein include the brachiocephalic trunk (BCT), left common carotid artery (LCCA), and left subclavian artery (LSA). The BCT sequentially branches to the right common carotid artery (RCCA) and the right subclavian artery (RSA), which in turn gives rise to the right vertebral artery (RVA). The RVA ultimately leads to the right radial artery (RRA) that courses in the right forearm/hand. The left subclavian artery (LSA) gives rise to the left vertebral artery (LVA) and ultimately leads to the left radial artery (LRA), which courses in the left forearm/hand.

In accordance with the invention, systems and methods are described that can provide a range of information that can assist a physician in deciding about treatment approaches for patients undergoing endovascular cerebral procedures where access to the cerebral arteries is required. In particular, the invention provides information about the degree of difficulty in accessing a brain vessel and the risk of complications while doing so via a radial artery or femoral artery access route, thereby assisting the physician in deciding about the optimal access route. In various embodiments, the system allows the physician to simulate different access routes to compare the difficulty and risk of complications.

For the purposes of initial general description, FIGS. 1A and 10 show catheter access from a RA access point whereas FIGS. 1B and 1D are described showing catheter access from a femoral artery (FA) access point.

Each of the above vessels and its relationship with respect to a distal or proximal (parent or daughter) vessel, that is the branching between two vessels can be described in terms of an angle between the two vessels and the direction one is approaching that vessel branch point from that is, either an RA or FA access point.

Branch points relevant to accessing each of the above vessels via one or more branch points (referred to herein as B1-B6) are characterized by the access point and the proximal arteries (vessel towards the site of the access point prior to the branch point) and distal arteries (vessel towards the brain vessel, beyond the branch point) that define take-off angles (R (Radial) or F (Femoral) referring to the access point). Representative take-off angles are shown in Table 2. As shown, as the direction that one is approaching a vessel junction is different, the take-off angle for a vessel junction will generally be different depending on the access site, i.e. the direction from which the vessel is approached. In most cases, as EE moves through a branch point, the EE will have to pass through an unsupported vessel zone.

In addition, features of vessel curvature that define the degree of tortuosity are relevant in accessing each of the above vessels. There will usually be sections along a desired vessel path with various features such as curvatures, apex and inflection points, different take-off angles and distances between apex/inflection points as well as the number and length of unsupported vessel zones branch-points, c- and s-shaped curves, kinked or coiled vessels. All these features contribute to determining overall access difficulty for a desired access route.

Navigating EE through vessels with tight curves, and numerous apex/tight inflection points requires the EE to be flexible. In addition, the translation of forward force and torque from a catheter end (outside the patient) to the catheter tip (inside the patient, in the vessels) gets less, and less predictable with an increase in overall tortuosity. Thus, in most cases, the access route with lower curvatures and a fewer number of apex/inflection points and fewer unsupported vessel zones will be the one with the highest likelihood of success.

TABLE 2
Vessel Branch Points and Representative Take-Off Angles

| | | Proximal | | Angle | |
|---|---|---|---|---|---|
| | | Artery | | R = radial | |
| | | (relative to | | artery | Representative |
| Branch | Access | catheter | Distal | F = femoral | Take-off Angle |
| point | Point | system) | Artery | artery | Range (°) |
| B1; R | RRA | RSA | RVA | R | 60-120° |
| B2; R | RRA | RSA | RCCA | R | 40-80° |
| B3; R | RRA | BCT | Aorta | R | 40-120° |
| B4; R | RRA | Aorta | LCCA | R | 80-120° |
| B5; R | RRA | Aorta | LSA | R | 40-140° |
| B6; R | RRA | LSA | LVA | R | 150-180° |
| B1; F | FA | RSA | RVA | F | 90-150° |
| B2; F | FA | BCT | RCCA | F | 150-180° |
| B3; F | FA | Aorta | BCT | F | 40-120° |
| B4; F | FA | Aorta | LLCA | F | 80-120° |
| B5; F | FA | Aorta | LSA | F | 60-150° |
| B6; F | FA | LSA | LVA | F | 150-180° |

As shown in Table 2, variations in take-off angles are substantial wherein individual branch points and/or a combination of branch points can readily enable, interfere with or prevent access from a RA or FA access point.

In addition to the above, vessel diameter, atherosclerotic burden and other parameters may also be considered in assessing a degree of difficulty from a particular access point as shown in Table 3.

TABLE 3
Other Parameters

| Parameter | Description | Range | Note |
|---|---|---|---|
| Aortic Arch | The average | 35-50 mm | A smaller aortic arch radius in |
| diameter | diameter of the | | combination with particular B |
| | aorta. | | angles may increase access |
| | | | difficulty from both RA and FA |
| | | | access sites |
| Presence of | Exophytic | 1-15 mm | In the presence of protruding |
| protruding | calcified or non- | thickness | plaques, the risk of plaque |
| plaques in the | calcified plaques | | dislocation with subsequent |
| access route | that originate from | | occlusion of small arteries in |
| | the vessel walls | | the brain (i.e. iatrogenic |
| | and protrude into | | strokes) increases. |
| | the vessel lumen. | | |
| Degree of | Relative degree of | No | As vessel wall calcification |
| vessel wall | atherosclerosis | calcification | increases, characteristics of |
| calcification | and calcification | at all - | the affected vessels change |
| | of the vessel | vessel wall | including rigidity/compliance. |
| | walls. | calcifications | |
| | | measuring | |
| | | several mm | |
| | | in thickness | |

### Vessel Tortuosity

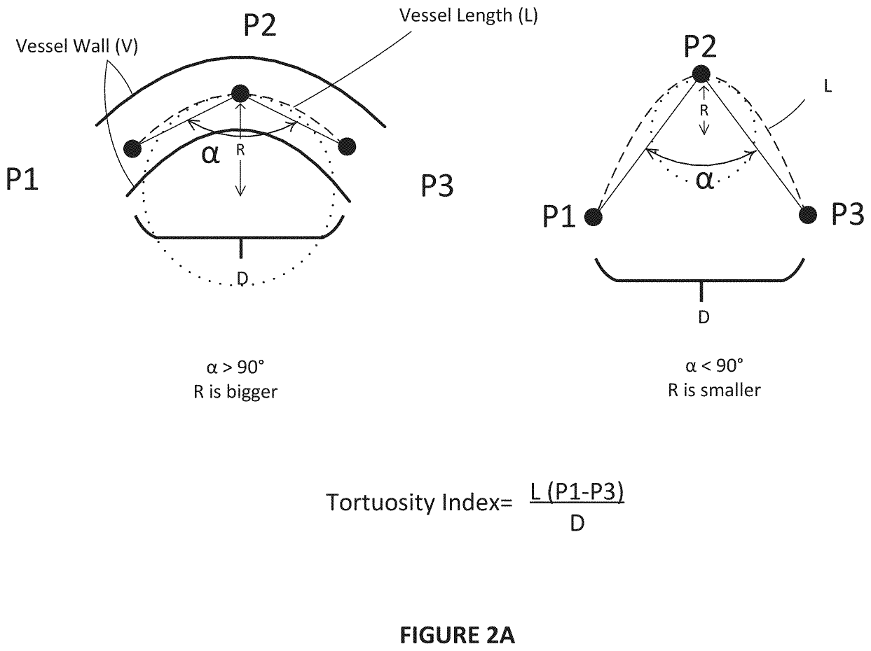

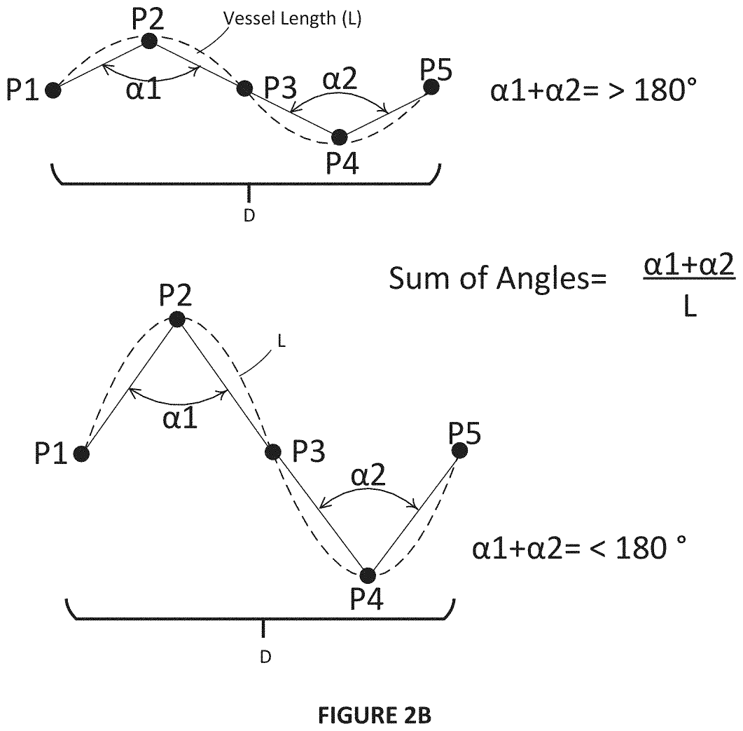

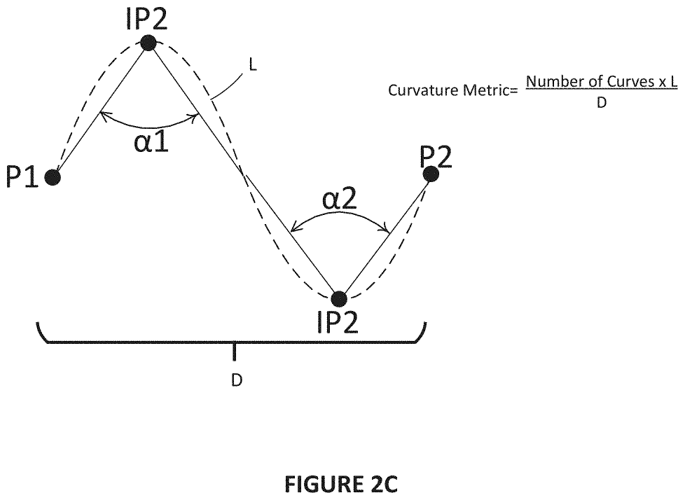

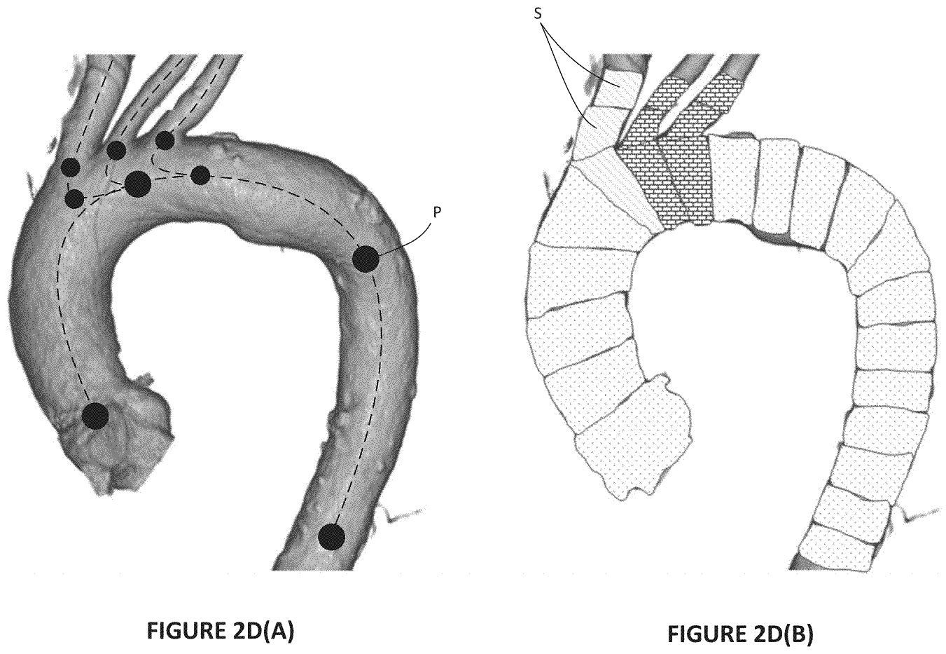

In various embodiments, as shown in FIGS. 2A-2C, vessel tortuosity can be measured by a number of quantitative measures including tortuosity index (TI), sum of angles method (SOAM) and Curvature metric (CM). FIG. 2A illustrates tortuosity index as the ratio between the length of the vessel and the straight-line distance between two end points. FIG. 2B illustrates SOAM as the ratio between the sum of angles at different inflection points and the length of the vessel between two end points. FIG. 2C illustrates curvature metric as the ratio of the number of curve apexes times the vessel length and the straight-line distance between two end points. In each case, a vessel V may be characterized by a number of points P between a start position P1 and an end position (generally P3 or higher) with intervening points representing apex or inflexion points (IPs) wherein the straight line connections between the points generally correspond to the central axis of the vessel. From each of the distances and angles between each point, the various tortuosity parameters can be determined. The start position and end positions may correspond to fixed anatomical points (FAPS).

FIG. 2D(A) and FIG. 2D(B) illustrate a 3D model of a patient's aorta and how FAPs and volume segments may be marked and/or calculated within vessels. As shown in FIG. 2D(A), FAPS P are shown being marked from the aorta valve to the descending aorta and at the junctions between the aorta and great cervical vessels together along the central axis of each vessel. FIG. 2D(B) illustrates how sections of vessels may be segmented into volumes as a means of defining available space for EE manoeuvres.

Quantitative measurement of tortuosity may be achieved according to the following general protocol:
a) Define fixed anatomical points/zones where tortuosity is to be measured as shown in FIG. 2D(A) and (B).
b) Measure the minimum distance between FAPS and the vessel length.
c) Determine and count the number of apex and inflection points between the FAPS.
d) Measure the angles at each apex, namely the maximum 2D angle in any 3D plane.
e) Calculate tortuosity scores such as:
   i. TI
   ii. SOAM
   iii. CM

As described below, identifying points/zones to be measured may be conducted manually or automatically via various algorithms including machine learning algorithms.

### Branch Point Navigation

Navigation of catheter systems through branch points can range from being relatively easy to difficult to impossible from different access points.

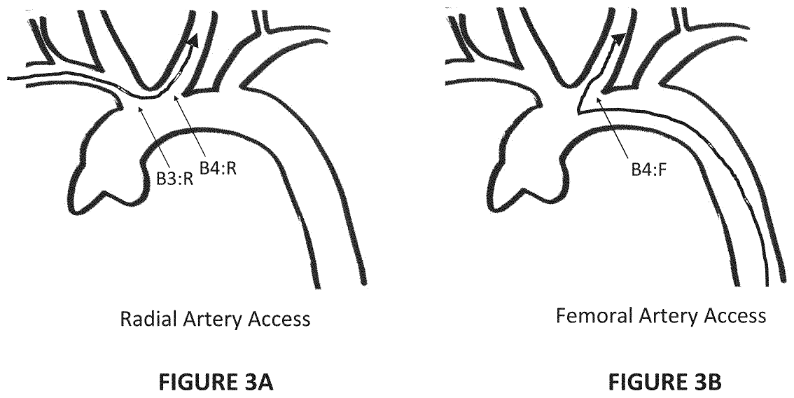

For example, FIG. 3A shows favourable B3:R and B4:R branch points and angles that would suggest access from the RRA to LCCA would be favourable as compared to the B4:F branch point angle when accessing the LCCA from a FA.

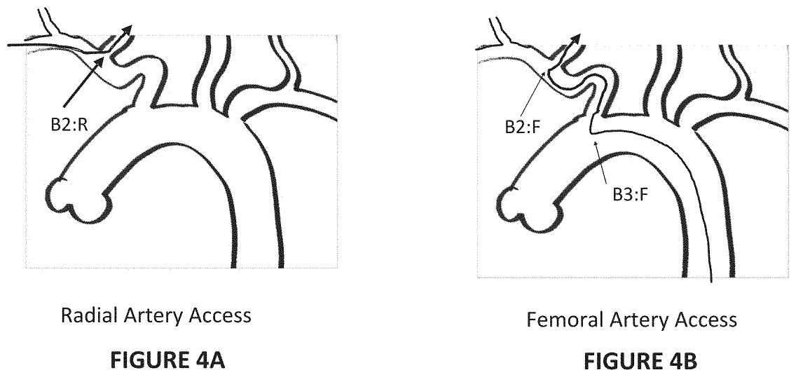

Similarly, FIG. 4A shows a favourable B2:R junction to access the RCCA that would suggest access from the RRA would be favourable as compared to the B2:F and B3:F junction angles to access the RCCA when accessed from the FA. Moreover, the situation shown in FIG. 4B shows a high degree of tortuosity in the BCT and would suggest a tendency for a catheter system to dislocate into the ascending aorta when coming from a FA access. Importantly, the anatomical examples described above are simple representative examples generally illustrating what could be the worst case or best case associated with a particular anatomy.

As such, it is readily understood that there are any number of intermediary examples where for example, it is unclear whether one route is better than the other and a decision may be based on a physician's qualitative feel of the overall situation. While experience may often enable a physician to make the right decision, less experienced physicians and those in training will not have the experience to make a decision based on qualitative feel of a patient's anatomy or be able to fully anticipate where problems may occur.

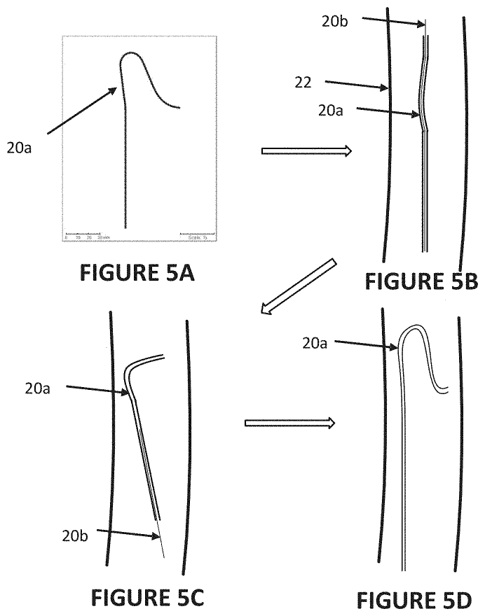

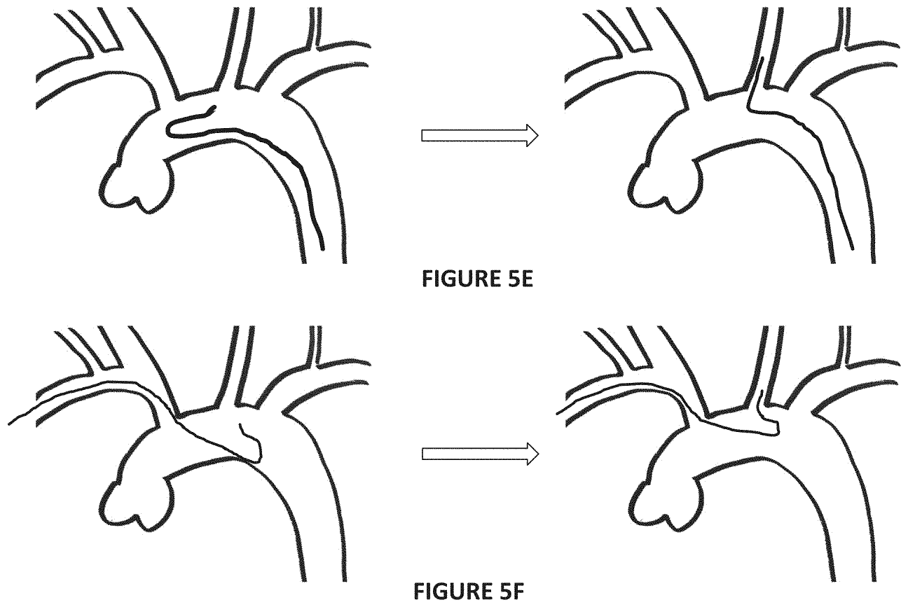

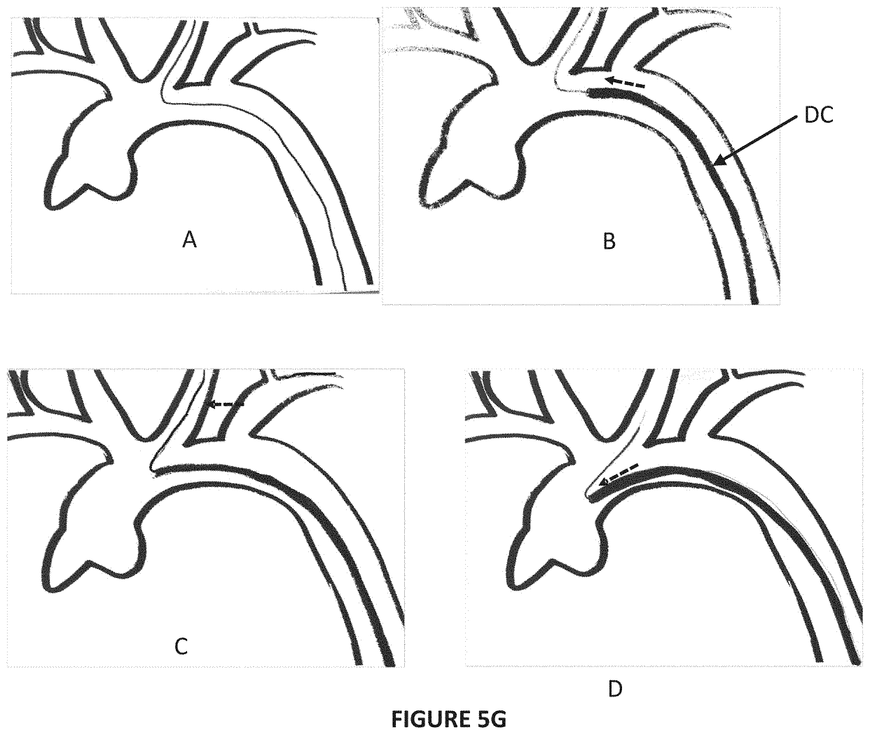

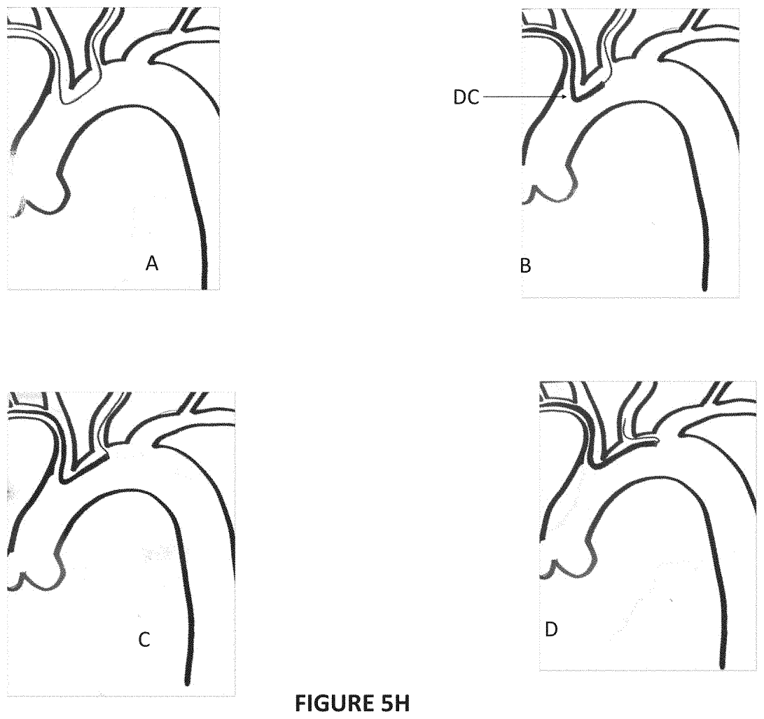

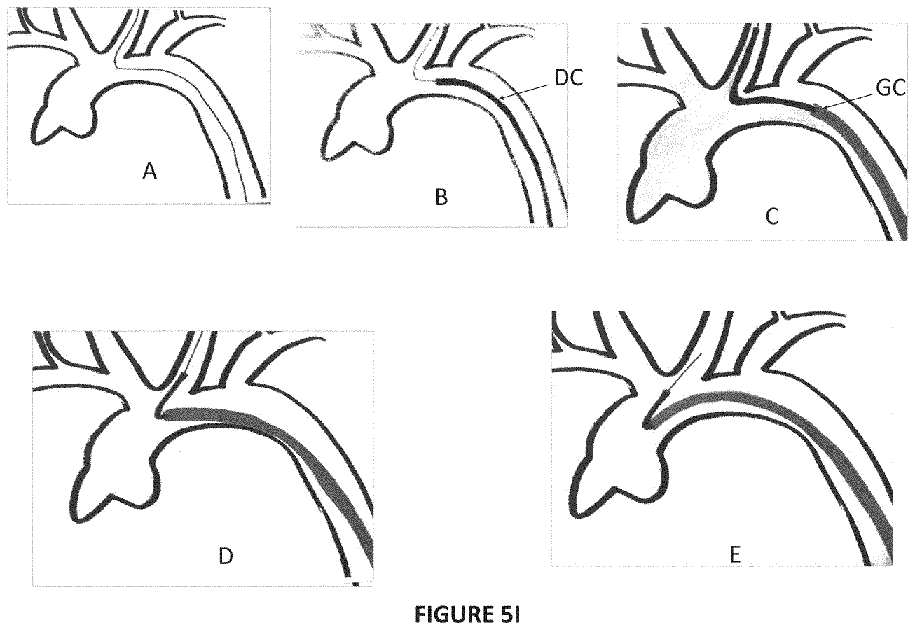

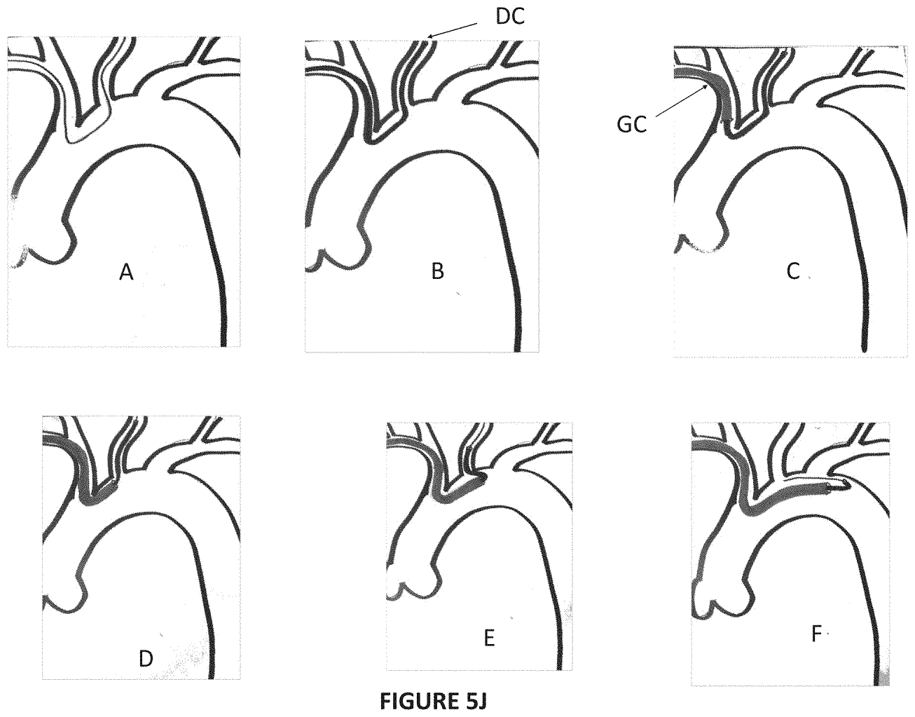

Generally, accessing cervical vessels from the RRA will often require navigation through branch points where the branch point angles are more acute and where a higher proportion of the overall route consists of unsupported vessel zones. For these procedures, the use of diagnostic catheters that have a “hooked”, i.e. a double-curved (“recurved”) catheter tip as the means of initially placing EE into a desired artery are required (FIG. 5A). There are many “hooked” diagnostic catheters available with varying lengths and curvatures, and several more are being developed. Generally, a hooked DC may be used 5% of the time when accessing the cervical vessels from a FA but may be used 90% of the time when accessing from a RA.

The use of a hooked DC poses various problems from a RA access point as explained below in describing a representative procedure of gaining access into the LCCA from the RA.

As shown in FIG. 5A, a hooked catheter, referred to herein as Simmons (SIM) DC 20a is characterized by a soft distal tip that is pre-formed into the shape of a hook. As such, in order to push this catheter through a vessel 22 from its access point, the hook shape is straightened with a guide wire (GW) 20b to enable its introduction and navigation from a distal vessel access point through the blood vessels of the arm to the aortic arch.

After gaining radial artery access, the SIM is assembled with the GW such that the GW protrudes a short distance from the tip of the SIM. This straightens the distal tip of the SIM allowing it to be advanced and steered through the brachial, subclavian and innominate vessels to the aorta as shown in FIG. 5B. When the distal tip of the SIM reaches the aorta (FIGS. 5C and 5D), the most common way to reform the shape of the SIM is: the GW is advanced to the descending aorta and subsequently the SIM such that the main curve of the SIM lies at the junction of the innominate artery with the aorta. Subsequently the GW is withdrawn to reduce the stiffness of the system. The SIM is pushed forward and without the GW the main curve of the SIM has a tendency to move towards the ascending aorta and form the SIM shape. This method is dependent on being able to get the wire into the descending aorta (this is not always possible). An alternative technique is that the GW is partially withdrawn and the hooked catheter spun around its own axis, such that the tip of the hooked DC is pushed in its pre-formed shape, thus providing a re-formed hook that has an approximate 180° bend and that can be used to select the origin of the target vessel. Re-forming the catheter by spinning it requires a large vessel and is usually done in the aorta being the largest artery of the body. As such, a variety of procedures may be undertaken including positioning the SIM in the ascending or descending aorta and/or utilizing the aortic valve (or left heart chamber) as a surface to deflect and position the DC into a favorable orientation to reform it.

Rarely, and only if the anatomy is favourable, the curve of the SIM can be reformed in the right subclavian or vertebral artery or the right common carotid artery.

The decision to re-form the hooked DC tip in the ascending or descending aorta and/or utilize the aortic valve/left ventricle as an abutment surface will depend on the patient's anatomy and generally whether or not the combined DC and GW can be navigated from a RA access point to the descending aorta. Whenever feasible, it is generally preferred to first try to utilize the descending aorta, then the ascending aorta and then the aortic valve as zones/surfaces for re-forming the hooked DC shape. This is because using the ascending aorta/aortic valve or left heart chamber as a surface to assist re-forming carries the risk of potential heart valve damage, embolization of plaques resulting in subsequent strokes and/or causing heart rhythm disorders.

It is generally also easier and quicker to re-form the hooked DC tip in the descending aorta and hence, will be preferred for a greater number of physicians as it generally requires less skill and experience. In contrast, reforming the SIM tip in the ascending aorta may be more difficult, take more time, increase the risk of complication and be more difficult to complete by less experienced physicians.

Once the DC has been reformed and regained its hooked shape, the physician will place the tip in a position to select the desired vessel origin and advance the GW and DC further into the vessel as shown in FIGS. 5E and 5F for different access routes. Manipulation of the GW and DC in combination with each other will enable further advancement. In situations where one or more take-off angles are particularly acute or when there are many unsupported vessel zones, it may become necessary to introduce GWs of different stiffnesses (typically less stiff) to carefully advance the DC and GW without dislocating the system out of the vessel being accessed, that is “losing access” by completely dislodging the DC and GW from the target vessel into the aortic arch so that the vessel has to be re-selected again. FIG. 5E shows the use of a SIM DC from a femoral access route and FIG. 5F shows the use of a SIM DC from a RRA access route.

FIG. 5G (A-D) shows how, when a wire has been positioned in the LCCA (5G (A)) from an FA access route, the advancement of a DC (5G(B)-5G(D)) can cause the wire to dislocate from the LCCA back into the aortic arch as the branch point into the LCCA is unsupported. Similarly, FIG. 5H (A-D) shows how when a wire has been positioned in the LCCA (5H(A)) from an RA access route, the advancement of a DC (5H(B)-5H(D)) can cause the wire to dislocate from the LCCA back into the aortic arch.

In addition, when a GW/DC has been introduced into a target vessel, it is also necessary that other catheters including guide catheters (GCs) can be positioned in the cervical vessels over the DC and GW. Accordingly, it is also important to ensure that once the GW has been positioned and the DC is advanced, that the GC does not dislocate the entire system as shown in FIG. 5I. FIGS. 5I(A-E) show the advancement of a GC from an FA access route to cause the GW/DC to prolapse from the system because the GC is too stiff and the GW too flexible. Thus, the GC is unable to follow the GW turn at the vessel branch point. FIG. 5H (A-F) show the advancement of a GC from a RA access route to cause the GW/DC to prolapse from the target vessel because the GC is too stiff and the GW too flexible. Thus, the GC is unable to follow the GW turn at the B4; R vessel branch point with an acute angle, at which the aorta gives rise to the left CCA.

### Decision Assist Tools (DATs) Overview

In accordance with the invention, decision assist tools (DATs) are described that can be activated or utilized at different stages of the overall treatment planning process to assist the physician in assessing:
What is the relative degree of difficulty accessing the target vessel for both routes?
Specifically,
   Can the target vessel be accessed in an appropriate time frame?
      via a RA route?
      via a FA route?
If the target vessel can be accessed by either route,
   What is the likelihood of success from the RA route insomuch as the RA route is preferred?
   What is the likelihood of success from the FA route insomuch as the FA route is preferred?
   If both an RA and FA are feasible, which route will be faster?
   If both an RA and FA are feasible, which route carries the higher risk of complications (e.g. microemboli producing silent diffusion hits)?
   Which approach has the lowest need for RMs?
   Are the potential RMs mild/moderate/severe?
   If a hooked catheter is required, where can the tip be reformed?
   What is the estimated degree of difficulty/risk of dislocating the system into the aortic arch when advancing the hooked catheter over the wire?
   What is the estimated degree of difficulty/risk of dislocating the system into the aortic arch when advancing the guide catheter over the DC and positioning the GC in the cervical vessel?
   What is the estimated degree of difficulty/risk of dislocating the system into the aortic arch when advancing additional EE that is needed to complete the procedure to the target vessel site?
   What are the procedure steps with the highest risk of dislocating the system from the cervical vessel to the aortic arch?
   Can a time estimate for a procedure be determined?
   What is the recommended equipment to access via either route?
      DC, GW, GC
   Is there a need for additional support to the guide catheter (e.g. quadraxial system) depending on the additional equipment being used intracranially or based on degree of tortuosity?
      Are there certain factors that would make one approach more unsafe (e.g. extensive protruding plaque in the descending aorta)

The DAT models are generally described as DAT1-DAT5 with increasing levels of information being provided or made available to the physician with each model and with increasing sophistication of simulating the performance of EE in a model vasculature. An “A” model utilizes expert opinion data, a “B” model utilizes a database consisting of data from past patients and a “C” model enables simulation of a procedure. That is, each DAT model can be built based on either or both of expert opinion providing input to the model and/or automatic review and assessment of data based on algorithms including machine learning algorithms and structural equation modeling.

In addition, in various embodiments, the prediction of success scores, likelihood of RMs being needed etc. are based on expert opinion (DATs 1A-4A; i.e. “A” designation). In other embodiments, once the past patient database has reached a sufficient size, the predictions can be based on a past patient database (DATs 1 B-4B; i.e. “B” designation). In other embodiments, once the past patient database has reached a sufficient size, the system may allow operators to practice either the whole procedure or critical procedure steps (DATs 1C-4C; i.e. “C” designation).

### Data Acquisition and Initial Processing

Data from past and current patients who have undergone a CT angiogram (CTA) of the aorta, neck and intracranial vessels can be a source of raw patient data for building 3D models of the relevant vasculature and be subject to analysis for quantifying vessel tortuosity. Magnetic resonance angiogram (MRA) may also be used as a raw patient data source. Typically, raw CTA images will be assembled and analyzed at a typical voxel size of 0.5 by 0.5 by 0.625 mm. Thus, 3D models built will enable measurements and parameter analysis at a high resolution. In various embodiments, 3D models may be further adapted to provide pulsatility information that includes vessel movement during the heart cycle.

Table 4 is a summary of the general functionality of each DAT model.

TABLE 4
Summary of DATs

| | DAT-1 | DAT-2 | DAT-3 | DAT-4 |
|---|---|---|---|---|
| Prediction | DAT-1a: | DAT-2a: expert | DAT-3a: expert | DAT-4a: expert |
| output | expert opinion | opinion | opinion | opinion |
| based on: | DAT-1b: past | DAT-2b: past | DAT-3b: past | DAT-4b: past |
| | patient database | patient database | patient database | patient database |
| | DAT-1c: | DAT-2c: provides | DAT-3c: | DAT-4c: |
| | provides the | the option of full | provides the | provides the |
| | option of full | simulation for the | option of full | option of full |
| | simulation for | entire procedure | simulation for | simulation for |
| | the entire | or specific | the entire | the entire |
| | procedure or | procedure steps | procedure or | procedure or |
| | specific | using | specific | specific |
| | procedure | information | procedure steps | procedure steps |
| | steps using | from DATs 2a | using | using |
| | information | and 2b | information from | information from |
| | from DATs 1a | | DATs 3a and 3b | DATs 4a and 4b |
| | and 1b | | | |
| Prediction | Advancing the | Advancing the | Advancing a GC | Advancing all |
| output: | GW and DC | GW and DC to | over the GW | the necessary |
| Estimated | to the target | the target vessel | and DC to the | EE that is |
| success | vessel | | target vessel | needed for the |
| score for: | | | | procedure over |
| | | | | the GC, GW |
| | | | | and DC to the |
| | | | | target vessel |
| Prediction | Potential low/ | Potential low/ | Potential low/ | Potential low/ |
| output | intermediate/ | intermediate/ | intermediate/ | intermediate/ |
| related to | high RMs | high RMs | high RMs | high RMs |
| RM: | encountered | encountered | encountered | encountered |
| | during the | during the | during the | during the |
| | procedure | procedure for | procedure for | procedure for |
| | | different | different | different |
| | | GW/DC | GC/GW/DC | additional EE |
| | | combinations | combinations | combinations |
| Accounting | No | Yes | Yes | Yes |
| for specific | | | | |
| devices? | | | | |

### 1^{st} Decision Assist Tool (DAT1)—General Success Scores Based on Vessel Anatomy and Expert Analysis

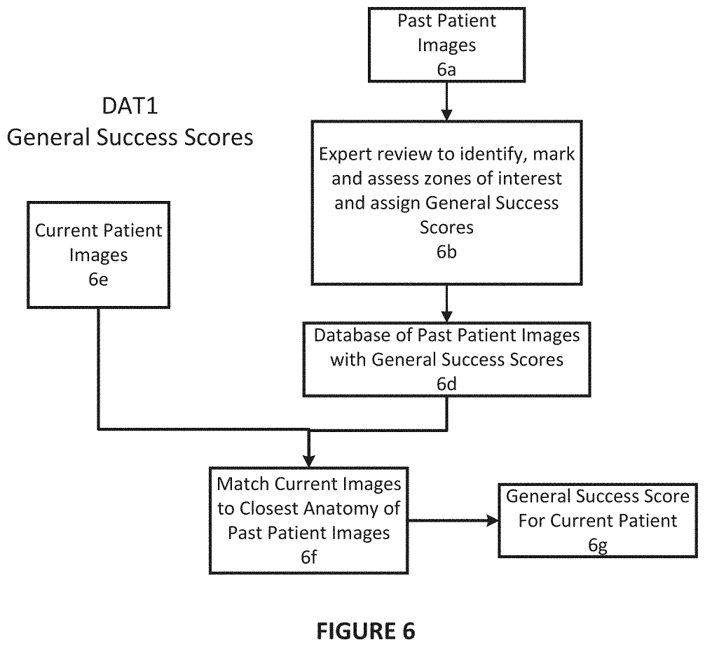

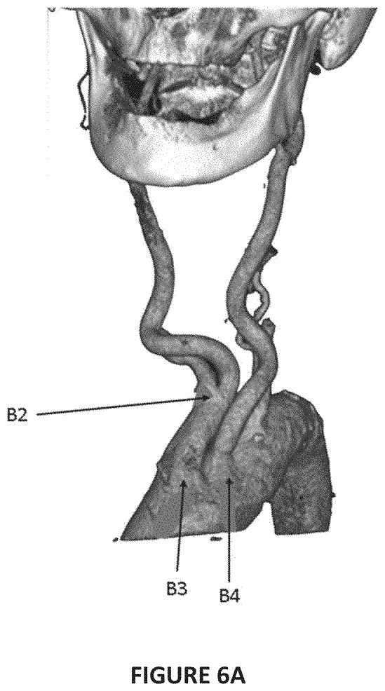

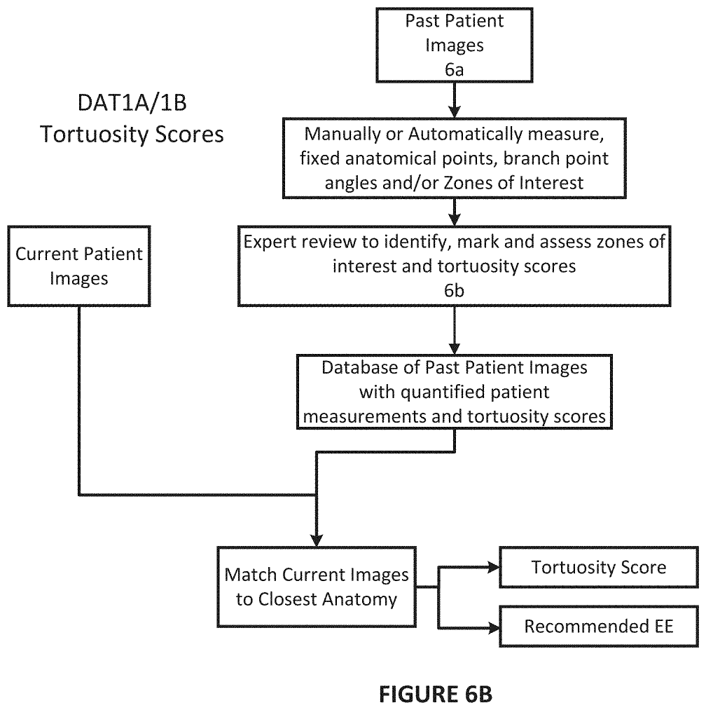

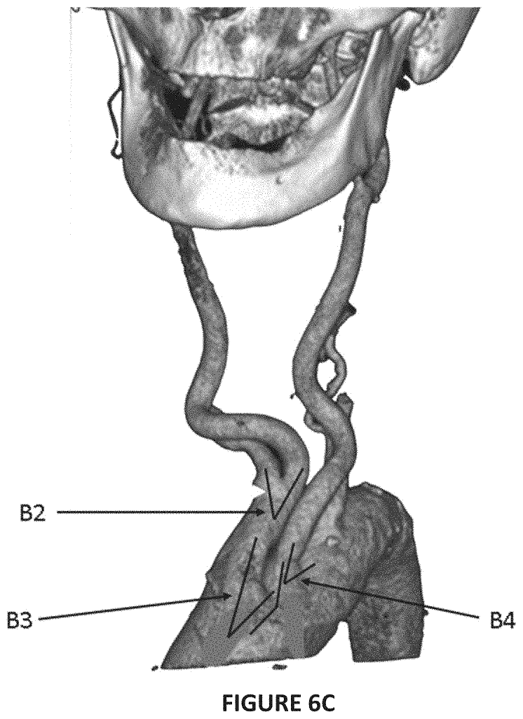

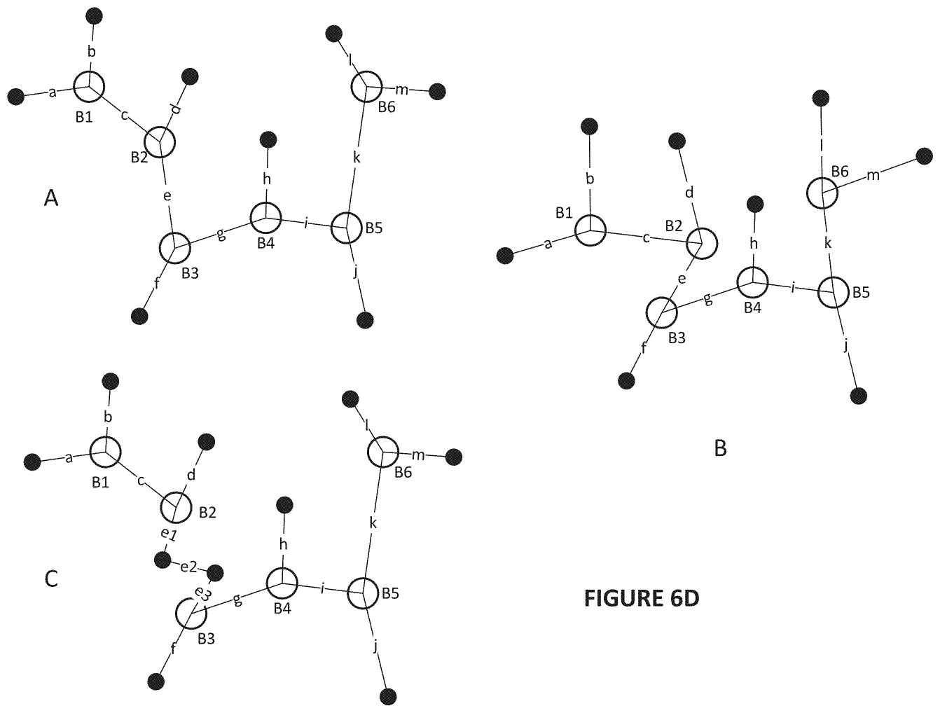

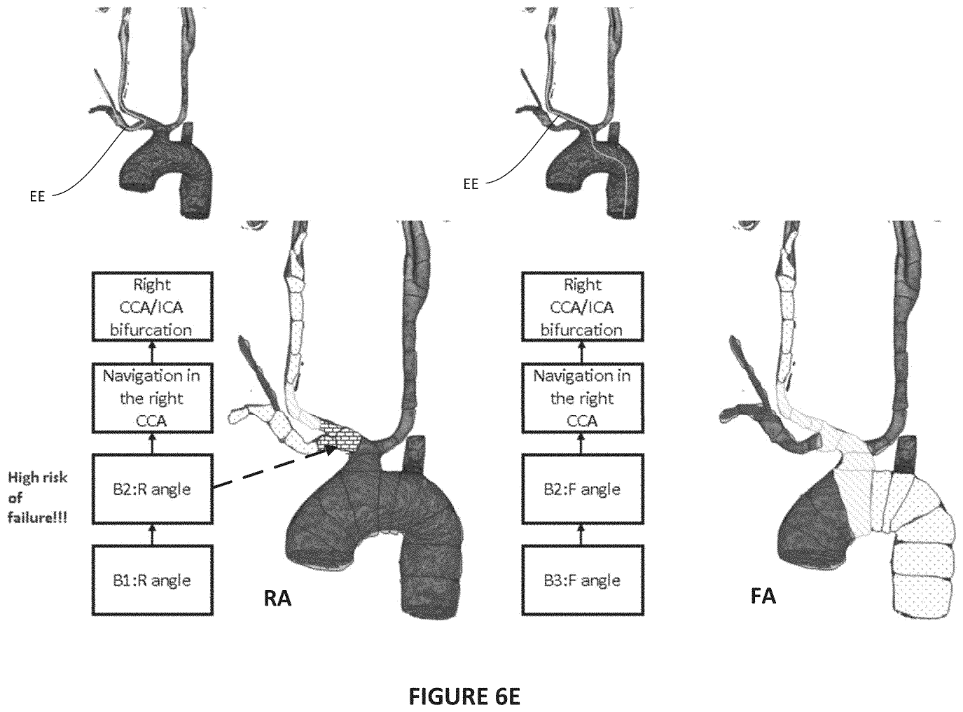

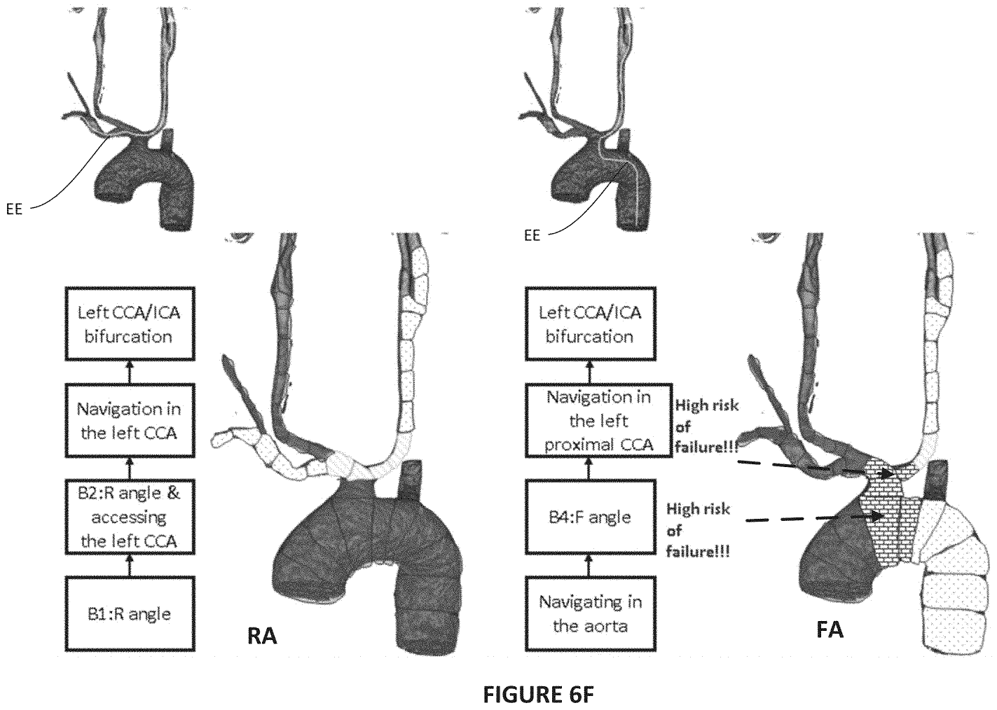

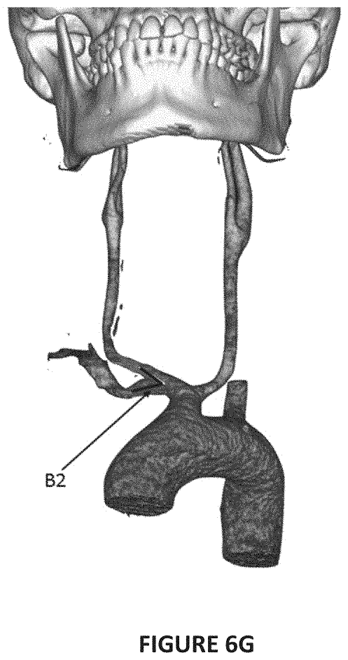

At a first level and as shown in FIG. 6, a DAT1 system is described that enables a comparison between images from a current patient to a database of past patient images in order to calculate general success scores for a current patient. From this comparison, a number of outputs can be provided to the physician that can assist the physician in decision making for the current patient and/or in training for endovascular procedures.

The past patient database includes a plurality of images from previous patients (patient records) 6a that may or may not have undergone endovascular procedures. Ideally, the patient records provide a representation of the scope of anatomies that an interventionist may encounter. The past patient database may include image data from patients of different sex, height, age and having a range of anatomical variations. Patient records 6a may also include associated data about past endovascular procedures undertaken and equipment used during those procedures. Such information may include information about procedures that may have been successfully or unsuccessfully completed.

Typically, images are available as a 2D layered model or a rendered 3D model as shown in FIGS. 3D and 6A that are used to assemble the database. After being made available, each model can be analyzed both qualitatively and quantitatively (6b of FIG. 6). For example, quantitative analysis can be conducted either manually or automatically to measure physical parameters including take-off angles, curvatures, apex points, unsupported vessel zones etc. and to calculate various tortuosity parameters including TI, SOAM and/or CM. Qualitative assessment can include qualitative data about the patient's anatomy, such as the presence of aortic wall calcifications or protruding plaques.

The initial measurements and analysis may be completed by expert review (DAT 1a) and include general success scores 6d for accessing each vessel by each route.

Current patient images 6e are compared to the past patient images to find the closest match 6f wherein success score information corresponding to one or more of the past patient images is displayed 6g. This information may be useful to the physician in making a treatment decision.

In further embodiments (DAT 1b), once a sufficiently large past patient database has been built which may include additional information regarding the success of completing procedures in a particular patient with particular EE, the classification of tortuosity, difficulty of navigating a catheter etc. can be based on analysis conducted on the past patient database rather than additional expert opinion.

In more advanced embodiments (DAT 1c), a full simulation environment based on information from earlier embodiments (DATs 1a and 1b) is offered to the operator, who can then practice certain procedure steps or the entire procedure prior to the “real” treatment.

That is, for each patient record, a series of images will have been analyzed by modelling software to a) assemble the images into a 2D or 3D model and b) enable measurements/analysis of relevant anatomical data. FIG. 6A shows a representative model of a past patient wherein images have been assembled into a 3D model of the patient's vasculature.

In one embodiment, after assembly of a model, one or more physicians would review the set of patient images and/or model from a patient case and would identify zones of interest to provide a subjective assessment of the likelihood of success for accessing particular vessels (as per FIG. 3D) as well as other relevant data including an estimated time to complete a procedure and/or recommendations for equipment and/or recommendations as where to reform a SIM catheter.

By way of example, the model shown in FIG. 6A is representative of tortuous BCT and LCCA access from the femoral artery. That is, in this patient, both B3 and B4 would be challenging to navigate from both RA and FA access. Hence, a physician reviewing this record might conclude that when a case presenting similar geometries is encountered that the likelihood of success to access the RCA or LCCA is less than 10% from the RA or less than 30% from a FA access. Additional information could be made including where an attempt to reform a SIM catheter should be made.

From this review, the record can be marked with general success scores 6d for getting past each branch point from each access point where general success scores will be numbers on a scale representing categories of relative difficulty.

Once assembled, the database with general success scores can be reviewed by users with or without a current patient. For example, a training physician may simply study the database to obtain both a qualitative and quantitative impression of the range of anatomies and the likelihood of success should he/she encounter similar real-case anatomies.

In addition, if the DAT is being used with a current patient and images from the current patient 6e, the DAT can be used to identify those records in the database that most closely match the current patient's anatomy.

This comparison may be done in a number of ways, for example, via groupings of past patient images into categories according to branch point angles, target vessels, access points or categories that are based on qualitative parameters around procedures.

As such, when a physician is reviewing a current patient and has identified a desired vessel (e.g. LCCA) and is looking to answer the question whether RA or FA access is possible, they may search the database and filter for records with similar anatomical features to the current patient.

Once a small number of past patient records has been identified whose characteristics may most closely match the current patient, those records may be examined more closely to review the general success scores of those past patients.

Thus, on review the physician may formulate a clearer picture of the likelihood of success from a particular access point to a particular target and base treatment decisions from that review.

As shown in FIGS. 6B, 6C and 6D, the past patient images may be reviewed with additional analysis to obtain/calculate additional data about each record.

For example, FIG. 6B shows additional refinement over general success scores and provides quantified patient measurements and tortuosity scores. In this case, for each patient record, the 3D model and/or the set of images would be reviewed by one or more physicians and each physician would mark particular anatomical points (e.g. vessel branch points) to enable calculation of quantitative measures of tortuosity. As shown in FIG. 6C, a physician may examine a 3D model and mark points that enable calculation of branch point angles. In various embodiments, this may also be done automatically (i.e. DAT1 B).

For example, after marking positions at B2, B3 and B4, the physician may note that that to gain access to the LCCA from either the FA or RA would be difficult due to the approximate 160 degrees branch point from the aorta to LCCA or from the BCT to the aorta. Alternatively, from the model, anatomical points may be estimated/calculated by the software, and tortuosity scores automatically calculated.

In addition, as shown schematically in FIG. 6D, a representation of patients' anatomies may be determined to enable comparisons between patients. FIG. 6D shows this representation for each main branch point in a normal anatomy (A), tortuous anatomy with acute branch point angles (B) and tortuous anatomy with multiple apex points between branch points (C).

As such, difficult branch points and/or tortuous sections between branch points may be identified, and specific points marked at positions within the section at the locations along the centerline for quantification.

For example, for a c-shaped curve as per FIGS. 2A-2C, P1, P2 and P3 may be marked on the centerline. The straight-line distance between P1 and P3 may be calculated as well as the centerline distance enabling calculation of TI. Similar marking/entry of points P1-Pn may be entered for more complex sections for calculation of other tortuosity parameters including SOAM and CM.

As shown in FIG. 6D, various patient anatomies can be mapped and the differences between anatomies quantified for the purposes of finding the best match to a past patient record.

In one embodiment, the current patient images are automatically assembled into a 3D model and the centerlines of vessels and segments automatically calculated as per FIG. 3D. Standardized intersection points between centerlines of different vessels may be used as primary branch points for comparison to past patients with similar branch points. For example, each of B1-B6 may be coarsely mapped in 3D space as shown in FIG. 6D (A-C). Angles may be calculated between branches and distances between branch points calculated. For example, for different patients A, B, and C, B1 angles “ab”, “ac”, “ca” and “cb” may be calculated as well the lengths of segments c, e, g, etc. These calculations may be repeated for all branch points. In addition, sections between branch points may be also be mapped to show apex points as shown in FIG. 6A(C) as e1, e2 and e3.

For example, if FIG. 6D(A) is a map of the current patient and FIG. 6D(B) and (C) are past patients, 6D(A) can be compared to the FIG. 6(B) and (C) maps in order that the relative difference between two patient records may be compared by particular angles and lengths.

By repeating the comparison across a plurality of patient records, one or more past patient record(s) most similar to the current patient can be identified.

Importantly, the ultimate selection of the “closest” record may include additional refinements.

Furthermore, when looking to match records, if with the current patient, it is known that the target vessel is the LCCA, data relating to non-relevant vessels may be ignored.

In various embodiments, the single best matched past images/data is presented and/or a ranking of the closest past images/data is presented.

Once the closest match to past patient image/data is determined (or the physician is presented with a ranked list), the physician can examine the assessments within that record(s). For example, the model may provide an output as shown in Table 4. Importantly, while the past patient data will likely include data from all branch point measurements, certain measurements may not be relevant to a procedure that is being planned for with the current patient. Hence, if the physician has entered the target, only relevant data may be displayed.

Data may also be output as shown in FIGS. 6E and 6F which compares RA and FA access routes for the same anatomy. As shown in FIG. 6E (RA), the B1:R and B1:R junctions/angle require navigation. The B2:R junction is particular acute, and may be marked as a high risk of failure. Such information may be illustrated to the physician in different formats including the steps required to complete the procedure and/or with a graphical representation of the anatomy where segments of the route are highlighted and color coded to show segments that may be easy to navigate (dotted segments), challenging (hashed segments) and difficult (brick segments). As shown in FIG. 6E, for the RA, the B2:R junction is marked as difficult or as having a high risk of failure whereas for the FA, the route has no difficult segments. The overall route for EE for each access point is also shown.

FIG. 6F shows an example where access from the RA is easier.

TABLE 5
Representative Comparison of Data from Current Patient and Past
Patient(s) for a procedure with the LCCA as target vessel.

| | Current | Past | | Noted |
|---|---|---|---|---|
| Parameter | Patient (°) | Patient 1 (°) | Correlation | Features |
| B1: R | 90 | 95 | High | |
| B2: R | 20 | 25 | High | |
| B3: R | 90 | 90 | High | plaque |
| B4: R | 120 | 140 | Low | |
| B5: R | 120 | 150 | Low | |
| B6: R | 160 | 150 | High | |
| B1: F | 90 | 105 | High | |
| B2: F | 170 | 150 | Med | |
| B3: F | 90 | 80 | High | |
| B4: F | 60 | 80 | Low | |
| B5: F | 120 | 115 | High | |
| B6: F | 160 | 170 | High | |
| Number of | None | 2 | Low | Noted and |
| apex points | | | | uncorrelated |
| encountered | | | | difference to |
| along the | | | | highlight |
| access route | | | | |
| . . . | . . . | . . . | . . . | . . . |

Output Scores

| | | | | |
|---|---|---|---|---|
| Proposed | Risks | Likelihood | Procedure | Procedure |
| Route | | of Success | Notes | Notes 2 |
| RRA to LCCA | Dislocation | 70-80% | Time-12 min | Time 13 min |
| Success | Risk at B4 | | EE- GW1 + | EE GW2 + |
| | Low | | DC(SIM2) | DC(SIM1) |
| | | | (DAT2) | (DAT2) |
| FA to LCCA | Dislocation | 80-90% | Time-10 min | Time 11 min |
| Success | at B4 | | EE- GW1 + | EE -GW2 + |
| | Very Low | | DC3a | DC3b |
| | | | (DAT 2) | (DAT2) |

EE is available from many manufacturers and in many different product forms. As such, different EE products that may be produced by different manufacturers may be referenced as “GW1” or “GW2”, etc. as generic descriptors of Guide Wires. Similarly, other EE such as Diagnostic Catheters may referenced as “DC1”, “DC2”, etc.

Thus, when the best match between the current patient's data and the past patient database has been made, the physician can examine the likelihood of success relating to access points and target vessels. From Table 5 above, if the target vessel is the LCCA, the past data shows a moderately faster procedure and higher success rate from the FA together with EE that has been used.

With this data, the physician can evaluate which access point may be preferable having regard to the likelihood of success and the possible time to complete. The physician may also make a decision based on their own skill level.

As understood by those skilled in the art, many combinations of data can be presented to the physician where the format and content of that data is derived from the granularity of data within the past patient database and the inputs that may be provided by the physician for the current patient. Importantly, with an appropriate level of granularity and filterable outputs, the physician has an objective basis on which to base a decision.

In various embodiments (e.g. DAT 1A), the process to obtain an objective assessment of past patient data may be refined by a standardized questionnaire/interview process of the expert physicians. That is, to obtain an objective assessment, during the review process as shown in FIG. 6, one or more expert physicians would be asked to answer a series of standardized questions about the case. Such questions could include assessment of the relative tortuosity of each branch from both an RA and FA access point to various target vessels. From the physicians' experience, the physicians may be asked to rank the difficulty of navigating a particular branch point on a scale of 1 to 5 with 1 being straightforward and 5 being very difficult/not possible. In the result, the database could include information that RRA to the LCCA would be ranked as “5-5” due to difficulty of the navigating B3:R and B4:R. Similarly, FA to the LCCA would be ranked as a 5 due to the difficulty of navigating B4:F. As noted above, this information may be output as shown in FIGS. 6E and 6F.

Notable curvatures, apex and inflection points, atherosclerotic burden, EE that is likely to work, time factors and other relevant questions may be also asked.

In other embodiments (e.g. DAT 1 B), once a sufficiently large past patient database has been built, the classification of tortuosity, difficulty of navigating a catheter etc. can be derived from analysis of the past patient database rather than expert opinion per as noted above.

If the images being reviewed by the physician experts show a generally average anatomy, the experts may conclude that for this average anatomy that access from both the FA and RA is relatively easy to the LCCA and rank the likelihood of success from both routes in terms of difficulty (e.g. Scores of 1) and how much additional time it would take from one approach relative to the other one. For example, both routes may be ranked as “straightforward” but the RA access route may have a +X minute time factor (e.g. +4 minutes) added. This may be particularly important information for the physician to consider, if the patient's condition is time-sensitive.

From the same patient images, the experts may reach different conclusions for accessing different vessels. That is, input provided for accessing the RCCA may determine that the likelihood of success from the RA may remain at greater than 90% (e.g. a 1 score) but the likelihood of success from the FA may drop to 70-80% (e.g. a 2 score). The reason that the experts may conclude that FA access drops (despite the patient having a generally “average” anatomy) may be presence of apex points in the BCT that would have to be navigated from the FA route but not from the RA route. FIG. 6G shows that RA to RCCA (B2) has a branch angle of less than about 20 degrees; hence access would be difficult whereas FA to RCCA (patient shown with bovine arch) would be straightforward once access to the BCT had been obtained.

In practice, the decision assist tool is useful in providing input to a physician after a decision has been made that it is worthwhile to conduct a particular endovascular procedure and the physician has determined the desired target vessel.

In various applications, the system can also be useful when a complete cerebral angiogram needs to be performed that requires access to both the right and the left carotid and vertebral arteries. In such a situation, if the system outputs a low success score for one of the four vessels from a RA route for example, the physician may decide to use a FA route, even if the remaining 3 vessels might be easy to access from a RA access route.

In various embodiments, the system is activated after non-invasive vascular imaging data has been collected from the current patient. That is, as soon as a non-invasive scan, for example a CTA or MRA, has been completed and while the physician is generally beginning their review of the current patient images, the system has completed or is concurrently conducting an analysis of current patient anatomy including take-off angles, vessel curvature, apex and inflection points of the vasculature.

As soon as the calculations have been made, the physician is advised and invited to input a desired target vessel. If all four cervical vessels need to be accessed as is often the case for the work up, for example in patients with acute sub-arachnoid hemorrhage, the physician can choose a “four-vessel option” to get an overall assessment for all four vessels as well as a separate assessment with potential points of difficulty for each vessel. The model receives that information and accesses the past patient database and calculates the likelihood of success (output) scores as described above.

Upon receiving the output scores, the physician can make a decision to conduct a procedure from a RA or FA or to take no action depending on the circumstances.

Utilizing fully modelled information from the patient's imaging data, expert input from DAT 1a and past patient data from DAT 1 b, DAT-1c allows a physician to plan for an intended procedure for example when a patient is being prepared for a procedure. That is, in one embodiment, the physician may input the desired vessel and available EE and the model determines if access to the desired vessel is possible using a generic selection of catheters.

In one embodiment, the DAT 1c model is connected to a neuroangiography operating console and allows physicians to practice and/or train in a realistic environment for single procedure steps or the whole procedure, by using a library of patient data, for example as part of a first “in vitro simulation phase” of their interventional training, prior to treating actual patients.

Additional functionalities can be provided in further embodiments as described below.

### Second DAT (DAT2)—Success Scores for GW/DC Navigation and Placement

In further embodiments, the DAT introduces functionalities that provide information about the likelihood for successful GW and DC placement in the target vessel within an appropriate time frame based on the specific GW and DC properties. In various embodiments, the DAT2 model expands upon the functionality of DAT1. As with DAT1, DAT2 assembles current patient imaging data and enables measurement of anatomical features and various tortuosity parameters within zones of interest (i.e. between various anatomical points as described above). The physician inputs target vessel information.

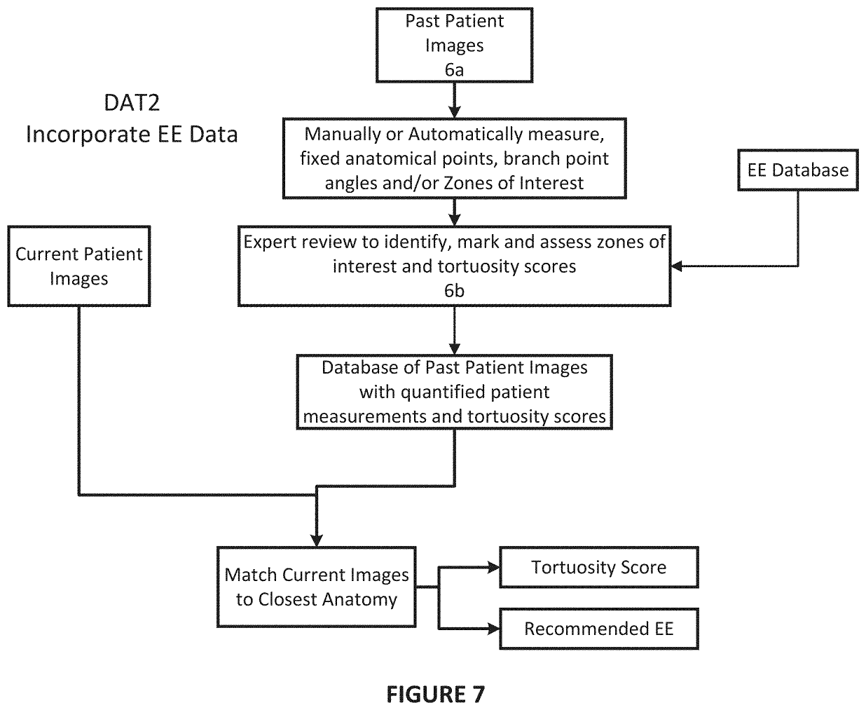

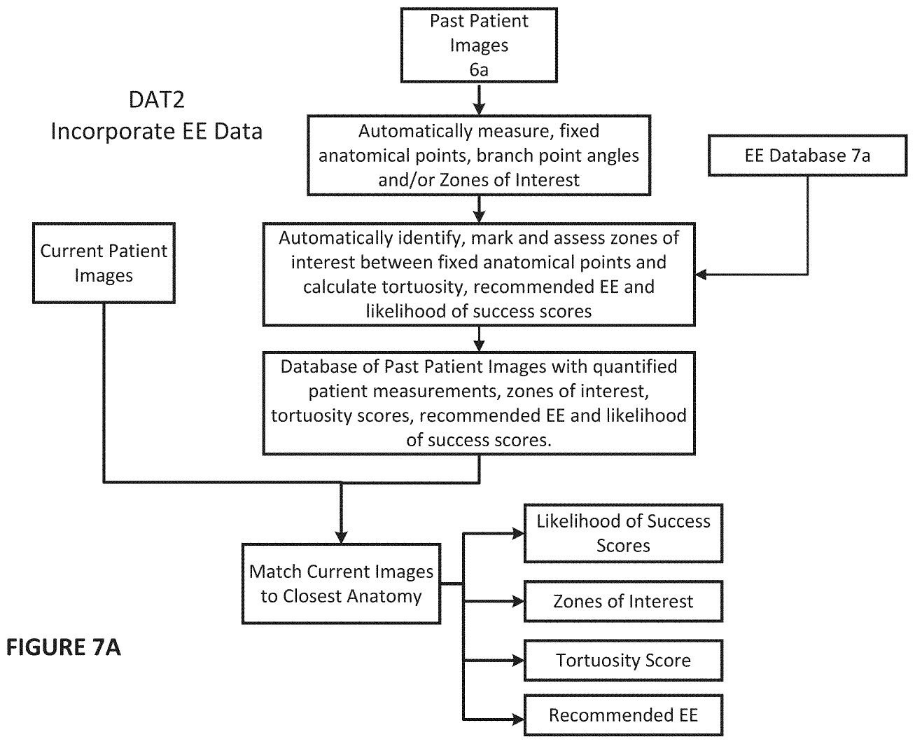

As with DAT-1, measurements and analysis may be completed by expert review (DAT 2A; FIG. 7).

In further embodiments (DAT 2B), once a sufficiently large past patient database has been built, the classification of tortuosity, difficulty of navigating a catheter etc. can be based on analysis of the past patient database rather than expert opinion per se. In various embodiments, classification may be a combination of both database analysis and physician input.

In more advanced embodiments (DAT 2c), a full simulation environment based on information from earlier embodiments (DATs 2a and 2b) is offered to the operator, who can then practice certain procedure steps or the entire procedure prior to the “real” treatment as described above.

Utilizing fully modelled information from the patient's imaging data, expert input from DAT 2a and past patient data from DAT 2b as well as information on properties of the selected GW and DC DAT-2c allows a physician to plan for an intended procedure with the selected GW and DC. The physician may input the desired vessel and available GW and DC he/she intends to use, and the model determines if access to the desired vessel is possible using the intended GW/DC combination.

In one embodiment, the DAT 2c model is connected to a neuroangiography operating console and allows physicians to practice and/or train in a realistic environment for single procedure steps or the whole procedure.

In addition, in one embodiment, the DAT2A model allows the physician to input data about recommended EE. In this embodiment, expert physicians may add recommendations for specific EE that may be utilized for a particular access route based on their observations of the patient's anatomy.

For example, with the LCCA as the target vessel, the physician may consider that as the time to complete an RA access procedure is longer and has a lower likelihood of success and that a FA procedure would be preferred in that the recommended EE (GW1 and DC3a) are on hand. However, the physician may decide based on this information that RA access is preferred because the recommended EE (GW1 and DC3a) are not on hand whereas the GW1+DC(SIM2) are on hand.

In another example, the experts may note that in considering access from the RA, a SIM catheter of a particular design may be more likely to succeed than others. The experts may note that a skilled practitioner should be able to access the relevant vessel in X minutes through the RA route and Y minutes through the FA route using catheter system A from the RA route and catheter system B from the FA route.

In one embodiment, the system accesses physical property data, e.g. data on stiffness, compliance and torquability of catheter systems stored in a catheter system database as shown in FIG. 7A.

The catheter system database includes physical property and performance data of individual catheters and combined EE systems from a EE database 7a explained in greater detail below.

In this embodiment, the DAT2 model evaluates the ability of EE such as GW and DC combinations to reach the target vessel(s).

Thus, the DAT2 model can output an estimated success score to gain access to the target vessel(s) via different access points utilizing specific combinations of GW and DC. For example, access from the RA to the BCT in a patient with a severely tortuous anatomy may be easier with a stiff GW and DC since they provide more stability and can “straighten out” the vessel. With a more flexible and thus less stable GW/DC system, access from the RA in the very same patient may be much more difficult.

The input may also provide specific information about any steps requiring “hooked” SIM type catheters.

For example, recommendations around SIM catheters may note one SIM catheter is soft at the point of the main curve and can be easily straightened out whereas a second SIM catheter is stiffer at the point of main curve and thus has a greater tendency to hold onto its shape. Both catheters will have advantages and disadvantages: the stiffer catheter may be better to hook the vessel and to advance wire; however, it may not advance over the wire.

Another issue with a stiffer catheter is that it may require more manipulation to regain its shape in the aorta and hence increase the chance of dislodging plaque. Thus, the DAT2 model may provide this output to the user.

Further still, in many hospitals, only a limited choice of EE is available. Thus, it is possible that, for example, access from the RA in a certain patient is generally feasible, but very difficult with the GW and DC equipment that are available at the physician's hospital. DAT2 allows the physician to restrict the GW and DC choice to the locally available EE and will provide the operator with information on how difficult various access routes when using the locally available DC/GW combinations.

Representative details EE properties included in an EE database are shown in Tables 6 (Diagnostic Catheters) and Table 7 (Guide Wires).

TABLE 6
Representative Diagnostic Catheter Properties stored in the EE Database

| Parameter | Description | Range | Note |
|---|---|---|---|
| Inner | Diameter of the | x-y | The inner diameter defines which |
| diameter | inner catheter | | GW can be used with the DC |
| | lumen | | |
| Outer | Diameter of the | a-b | The outer diameter defines the |
| diameter | outer catheter | | minimum vessel size that can be |
| | lumen | | accessed |
| Length | Total length of | c-d | The length has to be longer than the |
| | the catheter | | distance from the access point to |
| | | | the target vessel site. Some |
| | | | catheters may not be long enough if |
| | | | the aortic arch and cervical vessels |
| | | | are very tortuous |
| Shape | Shape of the | Single | Different catheter shapes are easier |
| | catheter | curved, | to use in a particular vessel |
| | | double | anatomy. In general, very curved |
| | | curved, triple | catheters are better to access |
| | | curved, | vessels with acute take-off angles, |
| | | recurved | while more straight catheters are |
| | | catheters | better to access vessels with take- |
| | | | off angles close to 180° |
| Material | Catheter | e.g. Dacrons, | The catheter material influences the |
| | material (may | Nylons, | thrombogenicity, and the degree of |
| | differ for | Silicones | friction with adjacent surfaces, i.e. |
| | different | | how well the GW glides in the DC |
| | catheter | | and how well the DC itself can be |
| | segments) | | navigated in blood vessels. |
| Compatibility | Compatibility of | — | Not every commercially available |
| other EE | the DC with | | DC is compatible with every GW |
| | GW, sheaths | | due to discrepancies in size, length |
| | and other, | | and hub systems, among other |
| | larger guide | | reasons. Only certain combinations |
| | catheters | | of DC and other EE can be used |
| | | | together. |
| Stiffness of | Stiffness of the | Very stiff, | Stiffer catheters have improved |
| the catheter | catheter main | stiff, | pushability compared to more |
| body | body | intermediate, | flexible catheters, i.e. when the |
| | | flexible, very | operator pushes the catheter end |
| | | flexible | outside the patient forward, this |
| | | | push is carried forward to the |
| | | | catheter tip nearly 1:1. However, the |
| | | | lack of flexibility of stiffer catheters |
| | | | can make it impossible to track |
| | | | acute take-off angles and thus |
| | | | prevent physicians from accessing |
| | | | the desired vessel in tortuous |
| | | | anatomy. |
| Stiffness of | Stiffness of the | Very stiff, | Catheters with stiffer tips are often |
| the catheter | catheter tip | stiff, | better to select (“hook”) vessels in a |
| tip | | intermediate, | tortuous anatomy. However, the |
| | | flexible, very | stiffer the catheter tip, the greater |
| | | flexible | the risk of vessel injury and |
| | | | dislocating the system. |
| Torqueability | Ease with | Very good, | Torqueing the catheter is necessary |
| | which the | good, | to re-form hooked catheters, and to |
| | catheter can be | intermediate, | navigate the catheter tip in order to |
| | rotated around | poor, very | select the origin of the target vessel. |
| | its own axis | poor | |

TABLE 7
Representative Guide Wire properties stored in EE database

| | | Variables/ | |
|---|---|---|---|
| Parameter | Description | Properties | Note |
| Diameter | Diameter of wire | varies | The wire diameter defines which |
| | | | catheters can be used with the |
| | | | wire |
| Length | Total length of | varies | The length has to be longer than |
| | the wire | | the distance from the access point |
| | | | to the target vessel site, and longer |
| | | | than the catheter used. Some |
| | | | wires may not be long enough if |
| | | | the aortic arch and cervical vessels |
| | | | are very tortuous |
| Shape of the | Shape of the | J-shaped, | Different wire shapes are easier to |
| tip | wire tip | shapeable | use in a particular vessel anatomy. |
| | | tip, etc. | In general, J-shaped tips are most |
| | | | commonly used but in certain |
| | | | situations shapeable tips that allow |
| | | | the physician to personalize the |
| | | | shape of the wire is more |
| | | | desirable. |
| Material | Wire material | Nitinol, | The catheter material influences |
| | (may differ for | stainless | the thrombogenicity, stiffness and |
| | different wire | steel | the hydrophilic character (i.e. how |
| | segments) | | well the wire glides when |
| | | | surrounded by fluid). |
| Compatibility | Compatibility of | | Not every commercially available |
| with other EE | the wire with DC | | wire is compatible with every |
| | and | | catheter due to discrepancies in |
| | microcatheters | | size, length and hub systems. Only |
| | | | certain combinations of wires and |
| | | | other EE can be used together. |
| Stiffness of | Stiffness of the | Very stiff, | Stiffer wires have improved |
| the wire body | wire main body | stiff, | pushability compared to more |
| | | intermediate, | flexible wires, i.e. when the |
| | | flexible, very | operator pushes the wire end |
| | | flexible | outside the patient forward, this |
| | | | push is carried forward to the wire |
| | | | tip nearly 1:1. However, the lack of |
| | | | flexibility of stiffer wires can make |
| | | | it difficult to track acute take-off |
| | | | angles and straighten out the DC |
| | | | curve. |
| Stiffness of | Stiffness of the | Very stiff, | Wires with stiffer tips are better to |
| the wire “tip” | wire tip | stiff, | pass tightly narrowed vessels, and |
| (typically | | intermediate, | “poke through” occluded vessels. |
| distal 5-10 | | flexible, very | However, the stiffer the wire tip, |
| cm) | | flexible | the greater the risk of vessel injury. |
| Torqueability | Ease with which | Very good, | Torqueing the wire is necessary to |
| | the wire can be | good, | re-form hooked catheters, and to |
| | rotated around | intermediate, | navigate the catheter tip in order to |
| | its own axis | poor, very | select the origin of the target |
| | | poor | vessel. |

Third DAT (DAT3)—Success Scores for Guide Catheter Placement after Successful GW/DC Placement

The DAT3 estimates the likelihood for successful GC placement in the target vessel within an appropriate time frame after the GW and DC have been placed based on the specific GW and DC properties. The DAT3 system expands upon the functionality of DATs 1 and 2. As with DAT1 and DAT2, DAT3 assembles current patient imaging data and enables measurement of anatomical features. The physician inputs target vessel information.

As with DATs 1 and 2, in one embodiment, analysis may be completed by expert review (DAT 3A).

In other embodiments (DAT 3B), once a sufficiently large past patient database has been built, the classification of tortuosity, difficulty of navigating a catheter etc. can be based on analysis of the past patient database rather than expert opinion per se.

In more advanced embodiments (DAT 3c), a full simulation environment based on information from earlier embodiments (DATs 3a and 3b) is offered to the operator, who can then practice certain procedure steps or the entire procedure prior to the “real” treatment.

Utilizing fully modelled information from the patient's imaging data, expert input from DAT 3a and past patient data from DAT 3b as well as information on properties of the selected GW, DC and GC, DAT-3c allows a physician to plan for an intended procedure with the selected GW/DC and GC. The physician may input the desired vessel, available GW/DC and GC he/she intends to use, and the model determines if access to the desired vessel and GC placement is possible using the intended GW/DC and GC.

In one embodiment, DAT 3c model is connected to a neuroangiography operating console and allows physicians to practice and/or train in a realistic environment for single procedure steps (e.g. placement of the GC after the GW/DC have been advanced) or the whole procedure (including GW/DC navigation and GC placement; “from beginning to end”.

Similar to DAT2, DAT3 accesses physical property data, e.g. data on stiffness, compliance and torqueability of GW systems stored in a catheter system database.

In addition to the features of DAT 1 and DAT2, DAT3 also accesses physical property data, e.g. data on stiffness, compliance and torqueability of GC/DC systems stored in a catheter system database.

The catheter system database includes physical property and performance data of individual GC catheters and combined EE systems that contain GCs explained in greater detail below.

The DAT3 model estimates the likelihood of success of a GC in combination with different GW/DC combinations to reach the target vessel.

Specifically, the DAT3 model may output an estimated success score to navigate the GC to the target vessel via different access points utilizing specific combinations of GW and DC.

In many hospitals, only a limited choice of EE is available. Thus, it is possible that, for example, access from the RA in a certain patient is generally feasible, but very difficult with the GW/DC and GC that are available at the physician's hospital. DAT3 allows the physician to restrict the GW, DC and GC choice to the locally available EE and will provide the operator with information on how difficult various access routes are and how high the risk of dislocating the GW/DC into the aorta is when the GC is advanced (see FIGS. 5I and 5J). This may be simulated for all locally available DC/GW GC combinations.

Fourth DAT (DAT4)—Success Scores for Placement of Additional Equipment that is Needed after Successful GW/DC and GC Placement

The DAT4 system expands upon the functionality of DATs 1, 2 and 3. As with the previous DATs, DAT4 assembles current patient imaging data and automatically measures anatomical features. The physician inputs target vessel information, the available EE and the model identifies combinations of GW/DC and GC that is available to access to the target vessel. The DAT4 model may further assess the likelihood of success of positioning additional EE that is needed for the procedure at the target vessel site within an appropriate time frame (e.g. stent-retriever in case of mechanical thrombectomy [fast placement required because of the time-sensitive nature of the disease], coiling catheter in case of an elective aneurysm coiling [time-insensitive procedure]), and the risk of dislocating the EE system from the target vessel into the aortic arch.

In various embodiments, the model may also take input from the physician regarding degree of stability of the GC that is needed once it is in position and is being used to advance other EE through it to proceed with the intervention. In general, if stiffer intracranial catheters and devices the operator intends to use are, and the more tortuous the vessels through which they are to be navigated, the more stable the GC needs to be. The physician can estimate the degree of stiffness of the EE to be used and the degree of intracranial tortuosity and input this estimate to DAT4. DAT4 can then calculate based on access route, GC GW and DC chosen, stiffness of the EE intended to use, and tortuosity, whether the GC would fulfill that degree of stability or not when the EE is advanced.

For example, the DAT4 model may show that access to a vessel is achievable using different combinations of DW and DC and GC to gain access to the cervical/cerebral vessels. However, as noted above, having gained access to a cervical/cerebral vessel does not ensure that additional EE can be advanced without causing the GW, DC and GC from dislocating out of the vessel and “falling back” into the aortic arch when the additional EE is advanced (See FIGS. 5G and 5H for example). Similarly, removal of EE or access equipment (GW and DC) may also cause dislocation of the system.

With the DAT4 model, in one embodiment, the physician inputs that they wish to introduce a specific piece of EE, for example a stent-retriever, over a specific GW/DC/GC combination and the DAT4 determines that based on the combined stiffnesses of the GW/DC/GC and their position, whether or not introducing and navigating the stent-retriever would cause the GW/DC/GC combination to dislocate from the cervical vessel into the aortic arch.

In one embodiment, the DAT4 may select possible types of EE, e.g. certain types of stent-retrievers, from a library of EE that would or would not enable advancement to a specific position. Further still, the DAT4 may also highlight specific procedure steps with high risk of failure, i.e. steps during which dislocation of the system from the cervical vessel into the aortic arch is likely.

As with DATs 1-3, in one embodiment, measurements and analysis may be completed by expert review (DAT 4A).

In other embodiments (DAT 4B), once a sufficiently large past patient database has been built, the classification of tortuosity, difficulty of navigating a catheter etc. can be based on analysis of the past patient database rather than expert opinion per se.

In the more advanced embodiments (DAT 4c), a full simulation environment based on information from earlier embodiments (DATs 4a and 4b) is offered to the operator, who can then practice certain procedure steps or the entire procedure prior to the “real” treatment.

Utilizing fully modelled information from the patient's imaging data, expert input from DAT 4a and past patient data from DAT 4b as well as information on properties of the selected GW, DC, GC and additional EE, DAT-4c allows a physician to plan for an intended procedure with the selected GW/DC, GC and additional EE. The physician may input the desired vessel, available GW/DC, GC and additional EE he/she intends to use, and the model determines if access to the desired vessel and EE placement and usage is possible using the intended GW/DC, GC and EE.

In one embodiment, the DAT 4c model is connected to a neuroangiography operating console and allows physicians to practice and/or train in a realistic environment for single procedure steps (e.g. use of a stent-retriever to treat an intracranial occlusion after the GW/DC have been advanced and the GC has been placed) or the whole procedure (including GW/DC navigation, GC placement and stent-retriever navigation and placement; “from beginning to end”).

### Models and Methods for Assisting Decision Making

In each embodiment of the DATs (DATs 1-4), as noted above the initial objective is to provide information about the likelihood of success of a certain access route to the physician based on the current patient's anatomy, the desired vessel (DAT1), available and required EE (DATs 2-4) and to allow for in vitro simulation of the procedure in the specific patient anatomy incorporating all the information mentioned above (DATs 1 c-4c). As discussed, varying levels of information can be provided with progressive and greater granularity of information.

As noted, a particular objective is to provide information to the physician of the likelihood of success from a RA and FA access point and/or output regarding catheters/catheter systems, and possibly additional EE if it is required for the procedure, most likely to enable access and/or the relative degree of difficulty from a RA or FA.

Various methods of collecting and processing this information in accordance with various embodiments are described below.

### Assemble and Analyze Patient Data

As described above and shown inter alia in FIG. 1A, aortic arch radius, vessel diameter, information on take-off angles, apex and inflection points and several other tortuosity indices are determined from a patient's scan data.

3D models of the aortic arch and associated vessels are preferably obtained automatically from CT angiogram data and input into modelling software such as Syngo.Via (Siemens Healthineers).

In various embodiments, the following steps are undertaken:
Step A—Create 3D model of vessel walls
   For example, create a wire frame virtual structure (or other model such as a rendered surface model) of a 3D volume of the vasculature incorporating the aortic valve, ascending aorta, descending aorta (10 cm from apex of aortic arch) and each cervical vessel take-off including the cervical vessels themselves. As a typical CT angiogram does not go low enough to include the aortic valve, but as the aortic valve is typically in a similar location in different patients, this information could be extrapolated.
Step B—Analyze the wire frame model to:
   Determine each vessel center line and apex and inflection points.
   Determine intersection points between vessel axis lines at vessel take-offs
   Calculate take-off angles for each relevant vessel take-off at these intersections that are encountered in the course of the access route.
Step C—Input from physician regarding available access EE and additionally needed, other EE, as well as presence of protruding plaques, time-sensitivity of the procedure and degree of GC stability needed (optional).
   Receive information from the operator on which access EE (GW and DC) are available in his/her hospital.
   Receive information from the operator on which additional EE needs to be advanced to the target vessel site to complete the procedure (e.g. guide catheter, stent-retriever for clot removal, coiling catheter for aneurysm treatment, etc.).
   Receive information about the presence of protruding plaques in the course of the access route (yes/no).
   Receive information on the time-sensitivity of the procedure (e.g. mechanical thrombectomy: highly time-sensitive, elective aneurysm coiling: not time-sensitive).
   Receive information on the degree of GC stability needed (e.g. high, intermediate, low).
Step D—Input desired target vessel and end position within the target vessel.
   Receive information of desired end position (target vessel site) of the
EE from the physician.
   Calculate sequence of vessel take-offs, take off angles, apex and inflection points to access desired target site from each access point.
Step E—Access past patient database and calculate success score(s).
   Identify/filter past patient records having undergone procedures to access similar target vessel sites.
   Compare current patient anatomical features with each past patient record accessing the target vessel site via a RA and FA route.
   Compare current patient's anatomical features mentioned above of past patients with similar anatomical features.
      For example, if the target vessel is the LCCA from a RA access and the B1:R, B2:R, B3:R, B4:R and B5R take-off angles from the current patient are 90°, 50°, 75°, 80° and 100°, with 3 evenly spaced apex points over a 5 cm vessel length, with no protruding plaques in the course of the access route, and there are 10 records from past patients in which the take-off angles are within a 10° range of the above-mentioned angles, the number of apex points is identical and there are no protruding plaques, these 10 records are flagged as cases being very similar to the current case.
   Examine flagged records for success.
      For example, if 7 of the 10 flagged records showed that the end position was successfully reached and 3 showed that end position was not reached, a preliminary (primary) success score of 70% could be presented.
   Conduct detailed comparison of individual vessel take-offs.
      As the overall difficulty could be vastly different if one of the anatomical features is slightly different, individual success score may be calculated for each vessel segment by determining particular differences of the current patient segmental anatomical features to the features within the same vessel segment of past patients.

From the current patient and past patient data, the data is analyzed to present output success scores to the physician.

In various embodiments, analysis is based on a primary success prediction and secondary success prediction.

### Primary Success Prediction

The 1° (primary) success prediction success is hereby defined as safe and successful access to the relevant vessel in an appropriate time frame. This time frame would vary based on whether the underlying condition is time-sensitive or not. This 1° success prediction is determined by the percentage of procedures from the past patients' database that were successful from the same access point with similar anatomical features (i.e. allowing only for a small, pre-defined range of deviation from the current patient's features, e.g. take-off angles within a range of ±5 degrees of the current patient's take-off angles), taking into account the mechanical properties of the EE the operator intends to use.

### Secondary Success Prediction

The 2° (secondary) success prediction is determined by a weighted comparison of the current patient's anatomical features, including vessel curvature, tortuosity indices, inflection and apex points, branch points and take-off angles, with the anatomical features of the past patients database.

Note that, in contrast to the 1° success prediction, where all anatomical features had to be similar to the current patient, the 2° success prediction is performed on a per vessel-segment basis. A vessel segment is generally a uniform segment volume relative to a fixed anatomical position. For example, the aortic arch relative to the aortic valve can be sectioned into uniform segments each having a consistent length (relative to its central axis) and radius. Each segment abuts an adjacent segment at a particular angle thus defining an assembly of interconnected segments. Daughter vessels may be defined by similar lengths but will have smaller radii. Branch angles are defined by angles of segments relative to one another. It should be noted that other vessel segmenting methodologies may be employed.

This means that the number of similar cases in the past patient's database that are available may be different for each vessel segment. For example, there may be 10 cases in the past patient database with a B1:R take-off angle within the ±5% range of the current patient's B1:R take-off angle, but for the B2:R take-off angle, there might be 8 or 12 or any other number of cases in the past patient database within the ±5% range of the current patient's angle. This approach allows for utilization of all available information for each vessel segment.
All records in which the procedure was not successful are grouped together: unsuccessful group.
All records in which the procedure was successful are grouped together: successful group.
For a group of the past patient data, all anatomical features are summarized/averaged.
The current patient data is then compared to the unsuccessful group and the successful group for each vessel segment.
   If a current patient's anatomical features are within one standard deviation of the features of the successful group, this vessel segment is given a +1 weighting factor. (This number suggests that the anatomical features in this vessel segment are similar to the features of past patients in whom the procedure was successful).
   If a current patient's anatomical features in a vessel segment differ more than one standard deviation of the successful group, this vessel segment is given a weighting factor of −1. (This number suggests that the anatomical features in this vessel segment are different from the features of past patients in whom the procedure was successful)
   If a current patient anatomical features in a vessel segment are within one standard deviation of the unsuccessful group, this vessel segment is given a −1 weighting factor. (This number suggests that the anatomical features in this vessel segment are similar to the features of past patients in whom the procedure was not successful).
   If a current patient's anatomical features in a vessel segment differ more than one standard deviation from the unsuccessful group, this angle is given a +1 weighting factor. (This number suggests that the anatomical features in this vessel segment are different from the features of past patients in whom the procedure was not successful).
   The above is repeated for each vessel segment in the access route to the target vessel.
   To obtain an overall score, the weighting factors of all take-off angles are summated (2° success prediction).
   While the overall score (success prediction) provides an overall estimate for access success, the individual vessel segment scores indicate which angles may be particularly difficult to pass and help the physician to prepare in advance for such particularly challenging parts of the procedure. See FIGS. 6E and 6F.

In summary, in this embodiment, the model objectively calculates how well the current patient data correlates to past patient data when procedures have been successful and unsuccessful.

In addition, the system can highlight to the physician how the current vessel segments, and in particularly their curvature, inflection and apex points, branch points and take-off angles, differ from past patients with successful/unsuccessful procedures performed via the same access point with the same target vessel, and thus suggest which vessel segments are the ones with the highest risk of dislocation and/or access failure.

This forgoing may be applied to procedures performed from any access point, i.e. a right and/or left FA and/or RA access.

### Endovascular Equipment Database

As described above, Endovascular equipment (EE) generally includes various wires and catheters that are assembled into co-axial systems to gain access to various vessels and enable various endovascular procedures to be conducted. Generally, EE includes guide wires (GWs) and microwires (MWs), diagnostic catheters (DCs), microcatheters (MCs), aspiration catheters (ACs), guide catheters (GCs) and balloon guide catheters (BGCs). As such, different pieces of EE are variable in terms of specific physical dimensional properties and performance properties in different vessels. That is, the physical measurements of different EE as well as the materials from which individual EE is constructed result in the overall functional/performance properties of each piece of EE. Moreover, as is known, EE may be constructed to have different dimensional and material properties along their lengths to enable specific functional properties in different zones to enable navigation through different levels of the vasculature and to provide certain functionalities (e.g. aspiration).

For example, diameters, wall thicknesses and materials of construction can be varied from a proximal end to a distal end of a catheter.

For example, a guide wire manufactured from a specific alloy with a consistent diameter along its length will have consistent properties including stiffness and torqueability in all zones of the wire. That is, all locations of the wire along its length will have a consistent minimum bending radius along its length.

Similarly, a microcatheter having a consistent material and wall thickness along its length will also have a consistent minimum bending radius along its length. If either the wall thickness or material is varied along its length, the stiffness (i.e. minimum bending radius) will vary along the length. The torqueability may also vary.

An assembly of a guide wire and microcatheter will also have different properties as compared to the individual components. For example, the distal tip of a microcatheter with a guide wire protruding 1 cm from the distal end of the MC will have different properties compared to the distal tip of an MC with a GW having a distal tip 1 cm proximal to the distal tip of the MC.

Thus, depending on the individual and collective properties of various pieces of EE, overall behaviour of catheters and catheter systems can be modelled with varying degrees of granularity. Greater granularity of modelled performance may be desired for the more distal regions of a catheter where physical dimensions of the vessels in which such pieces of equipment are interacting become smaller.

In one embodiment, a database of EE is assembled containing dimensional and functional data of individual pieces of EE and assemblies of EE for use in conjunction with 3D model data of a patient's vessels to predict the performance of EE within specific vessels. Table 8 shows representative data of a modelled guide wire and Table 9 shows representative data of a modelled microcatheter.

TABLE 8
Representatlve Data of Modelled Micro Wire

| Distance | | | | Min | |
|---|---|---|---|---|---|
| (from | | | | Bending | |
| proximal | | Diameter | Wall | Radius | Torquability |
| end) (cm) | Material | (inch) | thickness | (mm) | Factor |
| 1 | Stainless | 0.014 | NA | 5 | 1 |
| | steel | | | | |
| 2 | Stainless | 0.014 | NA | 5 | 1 |
| | steel | | | | |
| . . . | | | | | |
| 150 | Stainless | 0.014 | NA | 5 | 1 |
| | steel | | | | |

TABLE 9
Representative Data of Modelled MC

| Distance | | | | Min | |
|---|---|---|---|---|---|
| (from | | | Wall | Bending | |
| proximal | | Diameter | thickness | Radius | Torquability |
| end) (cm) | Material | (inch) | (inch) | (mm) | Factor |
| 1 | PU (with | 0.03 | 0.006 | 3 | 1 |
| | braiding) | | | | |
| 2 | PU (with | 0.03 | 0.006 | 3 | 1 |
| | braiding) | | | | |
| . . . | | | | | |
| 137 | PU | 0.026 | 0.005 | 2 | 0.5 |
| 138 | PU | 0.026 | 0.005 | 2 | 0.5 |
| 139 | PU | 0.026 | 0.005 | 2 | 0.5 |
| 140 | PU | 0.026 | 0.005 | 2 | 0.5 |

Calculate Feasibility of Introducing a Catheter/Catheter System into RA and/or FA to Gain Access to Position Past Desired Junction

From these measured properties, the EE is assembled as EE virtual models that can be moved through a virtual model of a patient's vasculature such that the behaviour of the modelled EE and assembled EE is predicted in a specific modelled vasculature, for example within the modelled vasculature of a current patient in preparing for a procedure.

### Determine Recommended Location to Orient Catheter for Target Vessel Access

Importantly, as noted above, a key consideration of a RA route is the high likelihood of requiring a “hooked” SIM type catheter, which has to be re-formed in the aorta. Hence, in various embodiments, the system determines the likelihood of being able to reform the hooked catheter in the descending aorta, ascending aorta or elsewhere after it has generally been determined that the hooked catheter can reach these vessels from an RA access point.

That is, for a current patient anatomy, the system determines whether there is room/volume within the ascending or descending aorta (or elsewhere) to re-form the tip of the hooked catheter and whether the catheter can reach the ascending or descending aorta.

To do this, the following general steps are completed:
a) Calculate the “re-form” volume of the hooked catheter's distal tip and/or obtain this information from an EE database.
b) Compare the re-form volume to the vessel segment volumes of the 3-D model of the ascending and descending aorta of the current patient and decide based on known volumes if there is sufficient volume in the current patient vessels.
c) The system may take into account take-off angles both leading to the aorta and the target vessel to assist providing an assessment of the likelihood of success.

In one embodiment, the system determines the recommended location to re-form by comparing the current patient anatomy to data within a past patient database and make the determination by a comparison of take-off angles and volumes as generally described above.

In one embodiment, the system may virtually advance modelled EE through a model of the current patient vessels and based on that simulation determine the likelihood of success of completing access to the target vessel by examining the results of one or more combinations of EE moving virtually to the aorta, descending aorta and ascending aorta. To do this, modelled combinations of EE are advanced through the modelled vessels in a series of steps where with each virtual advancement, the feasibility of each step is tested by the limitations of the modelled EE. Torqueing steps may be included (and highlighted to the physician).

In various embodiments, for each step of advancement, the system may assign a score to that step including, yes, the step is possible, no, the step is not possible or yes, step is possible with the chance of success being approximately X % (e.g. 10%). This score may be utilized to determine the difficulty of the different procedure steps, and to bring to the physician's attention what the most difficult steps are in which problems may be encountered.

In one embodiment, the steps of virtually advancing the modelled EE through a modelled vasculature may include a graphical presentation to the physician.

In one embodiment, with each step of advancement, the step is graphically displayed and each step of advancement, color coded according to the degree of difficulty/likelihood of success, with the step score described above. For example, if a specific procedure step has a high chance of success, the modelled EE or modelled vasculature may be overlayered with a green color. Similarly, if a certain other procedure step has a low chance of success, the modelled EE or modelled vasculature may be overlayered with a red color. Yet another procedure step that has a chance of success near 50% may be displayed with a yellow color. See FIGS. 6E and 6F.

Besides the difficulty of the single procedure steps, an overall success estimate (1° and 2° success predictions) may be displayed to the physician.

This forgoing may be applied to procedures performed from any access point, i.e. a right and/or left FA and/or RA access.

### Gaining Access to Target Vessel and Assessing Risk of Dislocation

If the system determines that the EE can be oriented to enable selecting/placement of the EE in a target vessel, in various embodiments, the system will then determine if the GW/DC can be advanced into the target vessel without dislocation from the selected vessel into the aorta.

In these embodiments, the system introduces additional parameters to those discussed above in connection with the modelled vasculature and modelled EE.

In one embodiment, the modelled EE takes into consideration the stiffness/flexibility of larger GCs that may be advanced over a DC/GW combination, and additional devices that may be required for the procedure (e.g. stent-retrievers to remove a blood clot, or coiling catheters to treat an aneurysm). It could also take into account the tortuosity of intracranial vessels and degree of forward pushability that would be needed to reach the point of interest. In general, the higher the requirement of pushability, the more stable the GC has to be in the neck. For example, within the simulation a DC/MW may have been successfully advanced into the cervical vessels with the distal tips of this EE ultimately positioned in the upper neck. In conducting this step during an actual procedure, a physician does not want the DC/GW to dislocate from the cervical vessels as the GC is being advanced or when the DC/GW are being withdrawn after the GC has been positioned in the upper neck.

In one embodiment, the simulation is continued as described above in which step-wise advancement or step-wise removal of EE is simulated.

For example, the simulation may show that a specific combination of a DC/GW can be successfully maneuvered from the RA to the LCCA with chances of success for passing the B4:R take-off angle being at approximately 75%. The initial stages of the simulation may therefore be positive. However, in continuing the simulation, when it is desired to advance a GC over the DC/GW, the stiffness of the distal tip of a chosen GC may indicate that chances of successfully pushing the GC around through the B4:R junction/angle are close to 0%, because the stiffness of the DC/GW may cause. them to dislocate from the cervical vessels into the aorta when the GC is pushed forward.

As above, in one embodiment, the presentation of graphical information may include color-coded displays to indicate where a problem is likely to occur.

In all of the above, the simulations may also determine a time-estimate to complete a procedure from a particular access point and with particular EE. When simulated from different access points and using different EE, a time-comparison of different simulated procedures may be presented to the physician.

### Model Summaries

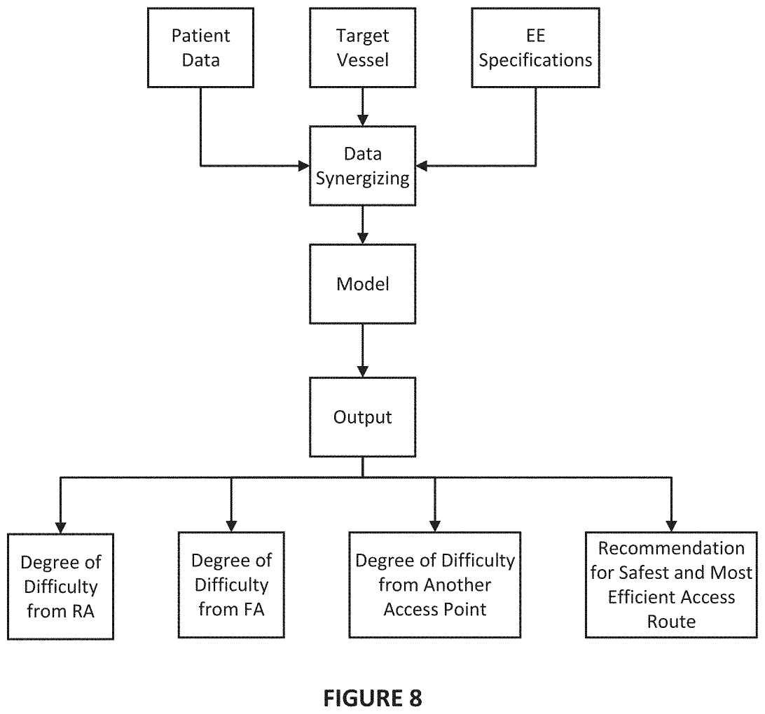

As shown in FIG. 8, the structure and objectives of the models are summarized including the model inputs, primary steps of assessing a difficulty score and the model outputs.

As described above, in various embodiments, various inputs may include EE specifications, the target vessel site and individual patient anatomy.

In the case of EE specifications, this information may be selected by the user via a graphical user interface such as a drop-down menu that includes information about specific commercial equipment used in endovascular procedures.

Similarly, a target vessel (including both vessel name and position within that vessel) may be input via a graphical user interface.

Individual patient anatomy may be input from existing imaging software and equipment such as via a DICOM communication protocol/interface.

Upon collection of each of the inputs, the data may be combined (e.g. the anatomical patient data may be combined with the EE and target vessel the surgeon has selected via the graphical user interface) and the combined data is then analyzed.

Generally, as described above, each model may undertake various analyses to assess one more difficulty scores. Various modelling methods may be utilized including “structural equation modelling”.

Structural equation modelling (SEM) is a mathematical method that allows for representation, estimation and testing of a network of complex relationships between variables, such as vessel tortuosity, vessel length and diameter, and the interplay between endovascular equipment and vessel anatomy. SEM includes measured variables, e.g. vessel length, and latent variables. Latent variables are variables that are not directly observed but can be inferred from other variables that are observed. In one example, the degree of difficulty when navigating through a particular vessel segment or the probability of a RM cannot be directly measured, but it can be inferred when combining information from several measured variables such as vessel tortuosity, branch point angles and EE properties. SEM includes path analysis and accounts for complex multi-directional relationships, mediation effects and interaction effects. For example, patient age influences atherosclerotic burden (with increasing age, atherosclerotic burden increases since vessels tend to calcify with age). Patient age also directly influences vessel wall fragility (with increasing age, vessel wall fragility increases because of degradation of the collagen and elastin fibers in the vessel wall). At the same time, atherosclerotic burden influences vessel wall fragility (vessels with atherosclerotic disease are more rigid and thus more prone to injury compared to non-diseased vessel segments). Thus, there is both direct effect of patient age on vessel wall fragility (patient age>vessel wall fragility) and an indirect effect of patient age on vessel wall fragility via atherosclerotic burden (patient age>atherosclerotic burden>vessel wall fragility). In other words, part of the effect of patient age on vessel wall fragility is mediated by atherosclerotic burden. Such complex relationships are very common in medicine, and SEM is able to accurately reflect them.

In various embodiments, these analyses can include assessing the degree of difficulty to:
a) form a hooked SIM type DC catheter;
b) gain access to a target vessel with a GW and DC;
c) advance a GC to a target vessel site; and,
d) advance additional EE to a target vessel site.

Upon completion of these analyses, various outputs may be presented to a user. These can include:
a) degree of difficulty of using radial access;
b) degree of difficulty of using femoral access; and,
c) degree of difficulty of using another access route (e.g. Direct carotid puncture, brachial or ulnar access).

Additional recommendations may be presented including:
a) a recommendation for the safest and most efficient access route;
b) estimated degree of difficulty for each procedure and each procedure step within a procedure for each access route;
c) recommended EE; and,
d) potential high-risk procedure steps/failure points.

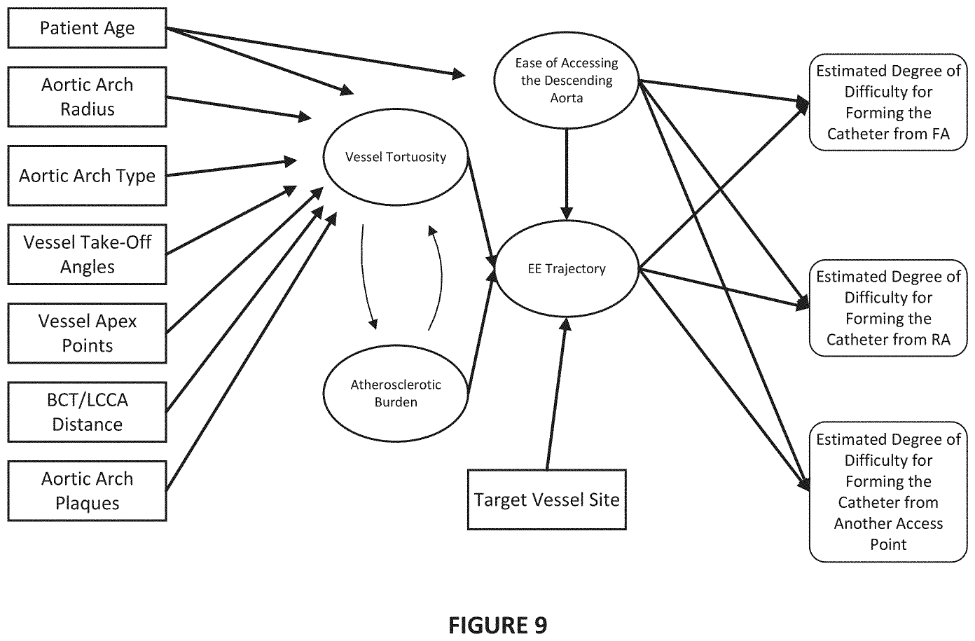

Examples of the various inputs, analyses and outputs are shown in FIGS. 9-10.

FIG. 9 shows representative input data, analysis and output for different aspects of various embodiments of the models and may specifically include a process for determining the degree of difficulty for reforming a hooked SIM type catheter in any of the various locations. In this embodiment, various patient specific data as measured from imaging are input to assess atherosclerotic burden and vessel tortuosity. From these determinations, ease of accessing the descending aorta and/or ascending aorta is determined to provide an assessment of difficulty from various access points to a specific vessel segment in which the SIM catheter can be reformed.

FIG. 10 illustrates a further embodiment in which past and present data are utilized by the system to improve the models wherein reinforcement learning models with iterative feedback loops are implemented to improve the ongoing accuracy of the system. For example, an interventionist may utilize the model to make a decision regarding a procedure. Depending on how that procedure went, the interventionist may provide feedback to the database at either or both a local hospital level or cloud-based level. The updated database(s) may be subjected to further analysis through an iterative feedback loop to provide updated models for subsequent use.

1. A system for analyzing tortuosity of a patient vasculature to provide input to a physician preparing for an endovascular surgical procedure, the system comprising:
a database having a plurality of past patient image records (PPIR) wherein each PPIR includes a visual representation of a patient's vasculature, the database enabling access and assessment by one or more experts and wherein each PPIR can be updated to include one or more success scores where a success score is a rating of difficulty for completing an endovascular procedure (EP) access from an access point to a target vessel.

2. The system as in claim 1 further comprising:
a current patient input system for uploading a current patient image record (CPIR);
a comparison system for comparing the CPIR to the plurality of PPIRs to determine a closest match between one or more PPIRs and the CPIR;
and a success score display system for displaying one or more success scores from the closest match.

3. The system as in claim 1 where the database includes separate success scores from two or more access points.

4. The system as in claim 3 where the two or more access points include a radial artery access point and a femoral artery access point.

5. The system as in claim 3 wherein the target vessels include any one of or a combination of right vertebral artery (RVA), right common carotid artery (RCCA), brachiocephalic trunk (BCT), left common carotid artery (LCCA), and left vertebral artery (LVA).

6. The system as in claim 1 further comprising a vessel measurement system, the vessel measurement system enabling measurement of vessel branch point angles from different access points.

7. The system as in claim 1 further comprising a vessel tortuosity measurement system (VTMS), the VTMS enabling measurement of zones of interest in a PPIR and CPIR and calculation of one or more tortuosity measures in a zone of interest.

8. The system as in claim 7 wherein the VTMS enables identification of one or more of vessel apex, vessel inflection point, vessel segment length, branch point angle, vessel support zone and vessel unsupported zone in 3D space.

9. The system as in claim 7 wherein the VTMS enables identification of one or more of vessel looping, vessel kinking, vessel coiling, vessel corkscrew in 3D space.

10. The system as in claim 7 where tortuosity measure includes sum of angles (SOAM), tortuosity index (TI), and curvature metric (CM).

11. The system as in claim 1 where the success score further includes a risky manoeuvre (RM) output score and where the RM output score includes any one of or a combination of a measure of risk of local injury to a blood vessel and risk of dislodging a plaque or thrombus.

12. The system as in claim 1 where the success score further includes a time factor score representing a time to complete an EP from any one of or a combination of a radial artery or femoral artery access point to a target vessel.

13. The system as in claim 1 further comprising a vasculature modelling system (VMS) enabling modelling of the vasculature in 3D space where the VMS utilizes PPIRs and/or CPIRs and where the VMIS determines 3D surface coordinates for vessel walls, vessel centerlines, branch point angles and vessel apexes.

14. The system as in claim 13 where the vasculature modelling system calculates tortuosity parameters for a modelled vasculature from the 3D surface coordinates for vessel walls, vessel centerlines, branch point angles and vessel apexes.

15. The system as in claim 1 where the system includes an endovascular equipment (EE) database and where each PPIR can be updated to include recommended EE to complete an EP from one or more access points to one or more target vessels.

16. The system as in claim 15 where the system includes a recommended EE display system for displaying an output of one or more pieces of EE recommended to conduct a procedure.

17. The system as in claim 16 where the EE database enables a user to filter for available EE at a treatment facility and update success scores based on available EE.

18. The system as in claim 17 where the system further comprises a hooked catheter reform module (HCRM) where the HCRM calculates vessel volumes within defined vessel segments and determines, based on physical size parameters of a hooked catheter, if the hooked catheter can be reformed in one or more vessel segments.

19. The system as in claim 15 where the EE database includes modelled parameters of EE and the system further comprises an EE advancement module (EEAM) enabling simulation of EE advancement within a modelled vasculature wherein modelled EE is progressively advanced within a modelled vasculature and the EE advancement module tests progressive movement of modelled EE within the modelled vasculature to determine if the modelled EE can be advanced based on the modelled parameters.

20. The system as in claim 19 where the EEAM includes an output module to display the feasibility of advancing specific EE within a vasculature.

21. The system as in claim 20 where the EEAM output module displays color coded zones within a modelled vasculature and where a displayed color represents relative feasibility of advancing EE through a zone of the modelled vasculature.

22. The system as in claim 15 where the EE database includes EE physical dimension and performance parameters for different EE, the EE selected from any one of a combination of guide wires, diagnostic catheters, guide catheters and stents.

23. The system as in claim 22 where performance parameters include any one of or a combination of stiffness and torqueability.

24. The system as in claim 22 where the EE database includes physical dimension and performance parameters for one or more combinations of guide wires and diagnostic catheters.

25. The system as in claim 22 where the EE database includes physical dimension and performance parameters for one or more combinations of guide wires, diagnostic catheters and guide catheters.

26. The system as in claim 25 where the EEAM evaluates the feasibility of advancing a guide catheter over a combined guide wire and diagnostic catheter based on a combined stiffness of each of the guide wire, diagnostic catheter and guide catheter.

27. The system as in claim 1 where the past patient database includes a questionnaire module enabling experts reviewing PPIRs to assign success scores to a past patient image record.

28. The system as in claim 15 where the system enables a training physician to access the PPIRs to review the success scores and EE used in past EPs.

29. The system as in claim 1 where each PPIR is assembled into a PPIR 3D model and the system further comprises a PPIR parameter measurement module for determining any one of or a combination of branch points, apex points, branch point distances and apex point distances.

30. The system as in claim 29 where each CPIR is assembled into a CPIR 3D model and the system further comprises a CPIR parameter measurement module for determining any one of or a combination of branch points, apex points, branch point distances and apex point distances.

31. The system as in claim 30 further comprising a comparison module where any one of or a combination of the branch points, apex points, branch point distances and apex point distances from the PPIR 3D models and a CPIR 3D model are compared in 3D space to identify one or more PPIR 3D models most closely matching the CPIR 3D model.

32. A system for analyzing a patient vasculature to assign a success score for completing an endovascular procedure (EP) from an access point to a target vessel, the system comprising:
a database having a plurality of past patient image records (PPIR) wherein each PPIR includes a visual representation of a patient's vasculature and success scores assigned to each PPIR;
a PPIR analysis module for calculating a success score for a current patient image record (CPIR) wherein the PPIR analysis module calculates branch point angles and vessel tortuosity between the access point and the target vessel of CPIR and compares the branch point angles and vessel tortuosity of the CPIR to branch point angles and vessel tortuosity of PPIRs to obtain a best fit to the current patient and assign at least one success score to the CPIR based on the best fit.
